(19) United States
(12) Patent Application Publication
(10) Pub. No.: US 2003/0157579 A1
(43) Pub. Date: Aug. 21, 2003

(54) Molecular sensors activated by disinhibition

(75) Inventors: Robert F. Balint, Palo Alto, CA (US); Jen-Horng Her, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: KaloBios, Inc., Mountain View, CA (US)

(21) Appl. No.: 10/076,845

(22) Filed: Feb. 14, 2002

(51) Int. Cl. A61M 36/14; A61K 51/00; G01N 33/53; G01N 33/537; G01N 33/543; A61K 39/395; A61K 39/42; C07K 2/00; C07K 4/00; C07K 5/00; C07K 7/00; C07K 14/00; C07K 16/00; C07K 17/00; A61K 38/00; C12N 5/06; C12N 5/16

(52) U.S. Cl. 435/7.92; 424/1.53; 424/143.1; 424/147.1; 424/159.1; 435/345; 530/300

(57) ABSTRACT

The current invention provides methods and systems for detecting the presence of a target molecule either in vitro or in vivo. The systems of the invention comprise interacting components, a reporter and a low-affinity inhibitor of the reporter, each of which is fused to a member of a binding pair. A target molecule that interferes with binding of the binding pair members can therefore be identified by detecting activation of the reporter molecule.

### BACKGROUND OF THE INVENTION

It is expected that a large fraction of the >100,000 gene products in the human proteome could eventually provide useful targets for therapeutic intervention in disease. However, the true targets will not be the gene products per se, but their functions, which invariably depend on interactions with other gene products. Thus, in the post-genomic era drug discovery strategies will focus on high-throughput screening for identification of inhibitors of key protein-protein interactions. Furthermore, the need for cell penetration and non-toxicity will place increasing importance on cell-based assays for high-throughput screening. Rapid, unequivocal identification of inhibitors of molecular interactions will require assay systems which produce positive signals upon inhibition of target interactions. The reason for this is that signal-to-noise and dynamic range properties of positive signal systems are invariably much better than negative signal, or signal reduction systems.

Unfortunately, few assay systems are currently available which can be adapted for positive signal detection of inhibitors of target molecular interactions. In most available assay systems, it is the interactions which produce the positive signal, and inhibitors of the interactions thereby inhibit the signal. For example, in the yeast two-hybrid system (Chien, C., Bartel, P., Stemglanz, R., and Fields, S. (1991) Proc. Natl. Acad. Sci. (USA) 88:9578-9582; Fields, S. and Song, O. (1989) Nature transcription factor subunits, which when brought together by the interaction mediate the expression of a reporter gene which confers a selectable phenotype on the cells. With such a system inhibitors of the target interaction can only be identified by their ability to abolish the selectable phenotype. When the selectable phenotype is cell viability, as it is in the preferred embodiment of the yeast two-hybrid system, the use of such systems for inhibitor selection is impractical.

Other cell-based assay systems which generate a positive signal upon the interaction of two proteins include enzyme fragment complementation systems (Pelletier J N, Campbell-Valois F-X, Michnick S W 1998. Oligomerization domain-directed reassembly of active dihydrofolate reductase from rationally designed fragments. Proc. Natl. Acad. Sci. USA 95, 12141-12146; Balint R, Her J-H, 1999, U.S. patent application Ser. No. 09/526,106), and enzyme subunit complementation systems (Rossi F, Charlton C, and Blau H M. 1997. Monitoring protein-protein interactions in intact eukaryotic cells by β-galactosidase complementation. Proc. Natl. Acad. Sci. USA 94, 8405-8410). In both of these systems host cells express interacting proteins which are linked to fragments or subunits of an enzyme. When the proteins interact the fragments or subunits are brought together, thereby reconstituting the enzymatic activity, which in turn confers the selectable phenotype on the host cells. Again, inhibitors of the interaction can only be detected by their ability to reduce or abolish the phenotype.

Positive signal inhibitor detection systems have many additional uses, including (1) epitope-specific selection of antibodies or other binding proteins from libraries, (2) affinity maturation of antibodies and other binding proteins, (3) identification of natural ligands of proteins of interest in expressed sequence libraries, (4) engineering enzyme activities for pharmaceutical and industrial applications, (5) analyte detection assays for clinical diagnostics, food testing, environmental testing, and process monitoring.

### BRIEF SUMMARY OF THE INVENTION

The current invention provides an improved reporter system, a “disinhibition” system in which an inhibitor interacts with a reporter molecule. The inhibitor and the reporter molecule are each conjugated to members of a binding pair. The interaction of binding pair members may be direct, or it may be mediated by other molecules. A target molecule that disrupts or interferes with the binding interaction between the binding pair members, a “disinhibitor”, can then be identified by the resultant increase in the reporter activity. (Disruption can occur when the target binds allosterically and causes a conformational shift that disengages the binding pair members. More often an activating target will activate by binding unbound binding pair members.)

This invention does not require the use of fragments of the reporter molecules. In particular, the invention further provides a system using an enzymatic reporter molecule and a low affinity inhibitor of the enzyme. The performance of the system may be enhanced when a high-affinity inhibitor is used and its affinity is “masked” by providing a low-affinity peptide sequence (the mask), which inhibits reporter-inhibitor binding only when fused to either the inhibitor or the reporter via a flexible linker. A reporter mask should block inhibitor binding without itself inhibiting the reporter. When a masked reporter or inhibitor is docked to the other by interaction of binding pair members, the low-affinity mask is displaced by the high-affinity interaction of reporter and inhibitor, and the reporter is thereby inhibited. Methods of making and using the disinhibition system are also provided.

In a preferred embodiment the reporter activation systems of the current invention comprise two interacting components, a reporter and low-affinity inhibitor of the reporter, each of which is fused to a heterologous protein that is a member of a binding pair. Thus, when the two binding partners interact, i.e., bind to one another, the inhibitor and reporter are brought into proximity, allowing the inhibitor to bind and inactivate the reporter. A target molecule that interferes with the interaction of the binding pair members also interferes with docking of the inhibitor, i.e., disinhibits the reporter molecule, thereby providing a selectable indicator of the presence of the target molecule. The interaction of the binding pair members need not be direct, but may be mediated by additional molecules, preferably one additional molecule.

One method of the invention provides a screening system for testing molecules for their ability to interfere with, or intercept the binding interaction between members of a binding pair, the system comprising: i) a reporter molecule linked to a first binding pair member, and ii) a low-affinity inhibitor of the reporter molecule linked to a second binding pair member; wherein, when the first and second binding pair members interact, the reporter molecule is inhibited; and further; wherein binding of a test molecule to a binding pair member displaces the inhibitor from the reporter molecule, or prevents the inhibitor from binding to the reporter molecule, thereby activating the reporter molecule. Test molecules may bind to either binding pair member. In an alternative embodiment, e.g., certain detection assays (see below), the test molecules may bind to only one of the binding pair members. Often, the reporter is an enzyme that confers antibiotic resistance on a host cell or makes a colored product, such as a β-lactamase. In one embodiment, the binding pair can be an antigen and an antibody or other binding protein such as scaffolded peptide or immunoglobulin variable region domain. Alternatively, the binding pair can be a receptor and a natural ligand that binds the receptor, or other gene products that finctionally interact in such cellular processes as signal transduction, gene expression, or metabolism. The assay may be performed in a host cell wherein the assay components are expressed from one or more vectors, or the assay may be performed in vitro with purified assay components. Host cells may include prokaryotes, for example, gram negative bacteria, or eukaryotes, for example, protozoa, yeast, plant, insect, nematode, or mammalian cells. The test molecules may be proteins such as antibodies or expressed gene products, expressed in the same host cells as the reporter components, but from a separate vector. The test molecules may also be any non-protein molecules which are made in the host cells, or which can diffuse into the host cells from the medium, or which can be mixed with the assay components in vitro.

In another aspect, the invention provides a sensor system and methods of using the sensor for identifying the presence of a target molecule in a sample (e.g., a clinical or environmental specimen. The sample is contacted to the sensor which comprises: i) a reporter molecule linked to a binding pair member, and ii) a low-affinity inhibitor of the reporter molecule linked to a second binding pair member, wherein binding of the target molecule to a binding pair member displaces the inhibitor from the reporter molecule or prevents the inhibitor from binding to the reporter molecule, thereby activating the reporter molecule. Often, the sensor employs an enzyme, such as a β-lactamase as the reporter molecule, and an inhibitor of the enzyme, e.g., β-lactamase Inhibitor Protein (BLIP; Strynadka et al. (1994) Nature 368: 657-660).

In practicing the method, the binding pair is often an antigen with an antibody, scaffolded peptide, or immunoglobulin variable region domain. Alternatively, the binding pair can be a receptor/ligand binding pair, or other gene products that functionally interact in such cellular processes as signal transduction, gene expression, or metabolism. In some embodiments, the contacting step is performed in vitro. In other embodiments the contacting step may be performed within a cell, or in vivo.

In another aspect, the invention provides a method of identifying a target molecule in a cell or population of cells, the method comprising: introducing into the cell or population of cells one or more expression vectors comprising nucleic acid sequences encoding a first binding pair member linked to a reporter and a second binding pair member linked to an inhibitor of the reporter; wherein the reporter is inhibited when the inhibitor is bound; culturing the cells or population of cells under conditions in which the first binding pair member linked to the reporter and the second binding pair member linked to the inhibitor of the reporter are expressed in the presence of a candidate target molecule, wherein a target molecule present in the cell population binds to a binding pair member, thereby displacing the inhibitor from the reporter, or preventing the inhibitor from binding to the reporter, and activating the reporter; and selecting a cell in which the reporter is active.

For some applications of the invention, e.g., affinity maturation, the selecting step comprises selecting a cell in which the reporter is more active than a reference standard of activity. For example, in affinity maturation, the first or the second binding pair member is an antibody and the candidate target molecule can be a random mutant of the antibody, which is selected by virtue of producing a higher reporter activity than that produced by the unmutated antibody when it is expressed as the candidate target.

The reporter systems of the invention have many uses, including (1) epitope-specific selection of antibodies or other binding proteins from libraries, (2) affinity maturation of antibodies and other binding proteins, (3) identification of natural ligands of proteins of interest in expressed sequence libraries, (4) engineering enzyme activities for pharmaceutical and industrial applications, (5) high-throughput screening systems for agonists or antagonists of protein-protein interactions involved in disease, and (6) analyte detection assays for clinical diagnostics, food testing, environmental testing, and process monitoring.

In a particular aspect, the invention provides a system and method of using the system for detecting a target molecule that interferes with a binding interaction between members of a binding pair, the system comprising: i) a reporter molecule linked to a first binding pair member, and ii) a tripartite conjugate molecule comprising a second binding pair member, an inhibitor of the reporter molecule, and an inhibitor mask having an affinity for the inhibitor such that: a) in the absence of a binding interaction between members of the binding pair, the mask constitutively binds to the inhibitor and prevents binding of the inhibitor to the reporter molecule; b) in the presence of a binding interaction between the first and second binding pair members, the reporter molecule displaces the mask from its binding site on the inhibitor, thereby inactivating the reporter molecule; wherein binding of the target molecule to the first or the second binding pair member prevents the inhibitor from binding to the reporter molecule and activates the reporter molecule. The inhibitor can be, e.g. a scaffolded peptide or an immunoglobulin variable region domain. In one embodiment, the mask is on the reporter molecule.

In some embodiments, the reporter molecule is an enzyme such as β-lactamase. In the case of a β-lactamase reporter molecule, the inhibitor can be a β-lactamase inhibitor protein (BLIP) or an enzymatically inactive β-lactmase mutant. The inhibitor mask can also be an enzymatically inactive β-lactamase mutant.

Often, the inhibitor mask is a peptide of between 3 and 12 amino acids. It can also be a scaffolded peptide such as a thioredoxin-scaffolded peptide.

In another aspect, the invention also provides a peptide mask for BLIP comprising any of the following sequences: ELRLTL, LT, LTPTVN, LTPVTI, LHTVGL, LTLHPT, LLTAAA, LTPT, or LTRSLP.

### BRIEF DESCRIPTION OF THE DRAWINGS

FIG. 1A. Reporter activation by target-mediated “disinhibition”. In this system one binding pair member is genetically fused to a low-affinity inhibitor of the reporter, and the other binding pair member is genetically fused to the reporter itself, such that the interaction docks the inhibitor to the reporter, and the latter is inactivated. Inhibitors of the interaction (Target Target Molecules) could then be identified by their ability to activate the reporter competitively.

FIG. 1B. Target-mediated disinhibition of β-lactamase. For an enzyme reporter, the low-affinity enzyme inhibitor preferably should have a Kₗ for the enzyme typically 10-100-fold higher than the optimal intracellular concentrations of the enzyme and inhibitor, so that the enzyme is 90%-99% active in the absence of an interaction. β-lactamase and its natural inhibitor BLIP interact with a K_{d} in the sub-nanomolar range. Thus, they interact constitutively at optimal concentrations in the cell. However, mutants of β-lactamase (E104K, or D, or Q, or A) greatly reduce this background non-specific inhibition. When these mutants are docked to BLIP by an interaction their effective concentrations with respect to one another rise at least 10,000-fold, so that enzymatic activity of each interaction complex is reduced by >90%. If the interactors are an antibody and its antigen, then the system may be used to select variants of the antibody with higher affinity by co-expressing the enzyme and inhibitor fusion proteins with a library of mutants of the antibody, and screening for enzyme activity which is higher than that produced by the unmutated antibody. Alternatively, antibodies with other properties may be selected. For example, if the parent antibody is from a mouse, then human antibodies for the same antigen could be selected by co-expressing the antibody/antigen-reporter/inhibitor fusion proteins with a human antibody library, and selecting for activation of the enzyme. The system may also be used to screen for inhibitors of any target interaction by co-expressing or exposing the interactor-enzyme/inhibitor fusion proteins, either in cells or in vitro, with candidate inhibitors, either singly or simultaneously, and screening or selecting for activation of the enzyme. Protein targets and their interactors may be complete or partial products of naturally-expressed sequences, peptides or scaffolded peptides, or antibodies. They may interact either directly or via additional molecules, which may be produced by the cells or added to the growth medium. The protein targets and their interactors are genetically fused to either terminus of the enzyme, inhibitor, or activator, via flexible peptide linkers comprised of typically 3-6 iterations of Gly₄Ser.

FIG. 2. Affinity maturation of an antibody by competitive activation of interaction-inhibited β-lactamase. The subject “low-affinity” antibody is genetically fused to either the carboxy-terminus or the amino-terminus of the β-lactamase Inhibitor Protein (BLIP) of Streptomyces clavuligerus (Strynadka et al., Nature 368: 657-660 (1994)) via (Gly₄Ser)₃₋₆ linkers. The antigen is similarly fused to either terminus of a variant of TEM-1 β-lactamase, such as the E104K mutant, which has a K_{d} for BLIP of 10-100 μM. When these fusions are expressed in the E. coli periplasmic space at concentrations which are at least 10-fold higher than the K_{d} of the antigen-antibody interaction, then the enzyme will be fully inactivated. If an additional gene encoding the same antibody unfused is expressed from a separate plasmid in the same cells at a level which is at least 10-fold lower than that of the fused antibody, it should cause no more than a ˜10% activation of the enzyme. Under these conditions any variant of the unfused antibody which has a higher affinity than the parent antibody will produce a greater activation of the enzyme, and will thereby confer on the cells a higher plating efficiency on restrictive concentrations of antibiotic. Successive rounds of replating will allow such variants to be enriched to the point that they can be cleanly separated from the parent and variants which do not have higher affinities. The same system may be used to select for other antibody properties, or for antibodies which compete with other interactions. For example, a mouse antibody which binds a desired epitope on an antigen may be used in the system to guide the selection from human antibody libraries of human antibodies which bind to the same epitope. Alternatively, a target receptor-ligand interaction could be used in the system to guide the selection of human antibodies which specifically interfere with ligand binding, and some of such antibodies may even mimic the signal transducing effects of ligand binding.

FIG. 3. Target-mediated disinhibition of β-lactamase using the wild-type enzyme and a mask for the inhibitor. The mask blocks the high-affinity interaction between BLIP and wild-type β-lactamase when the two are not docked to each other by the interaction of binding pair members fused to the masked BLIP and β-lactamase. When docking occurs, however, the higher-affinity BLIP—β-lactamase interaction displaces the low-affinity BLIP-mask interaction to achieve high-affinity inhibition of β-lactamase. The binding of target target molecules to either binding pair member prevents docking of the masked BLIP to β-lactamase, resulting in activation of the latter. The mask can also be placed on β-lactamase where it would protect β-lactamase from BLIP by binding to the enzyme with low affinity and without interfering with its activity.

FIG. 4. Epitope-guided selection of a human antibody which binds to the same epitope as a murine antibody with desired bioactivity.

FIG. 5. Expression vectors for the inactivation of β-lactamase by interaction of c-fos and c-jun leucine zipper helixes and for the activation of β-lactamase by competitive disinhibition by the c-jun helix. BLIP, β-lactamase inhibitor protein; clacp, constitutive mutant of the lactose operon UV5 promoter; SP, signal peptide for translocation across the plasma membrane into the periplasmic space; pBR322 ori, p15A ori, plasmid origins of replication which are compatible, i.e., both plasmids can co-exist in the same cell. f1 ori, bacteriophage f1 origin of replication (allows phage rescue); cat, chloramphenicol resistance kan, kanamycin resistance; tt, transcription terminator.

FIG. 6. Expression vectors for antigen-antibody interaction-mediated inactivation of β-lactamase and for antibody-mediated activation of β-lactamase by competitive disinhibition. Antibodies are expressed as Fabs (LC plus Fd) from dicistronic transcripts. IRES, internal ribosome entry site for re-initiation of translation on the downstream cistron. This embodiment includes, a “competitor” molecule, i.e., the Fab against which the “test” Fabs must compete for binding to the antigen in order to activate the reporter. The antigen in Example 2 is CD40ED, the extra-cellular domain of the human B-cell activation antigen CD40.

FIG. 7. Expression vectors for the selection and validation of low-affinity, cis-acting peptide masks for BLIP. Cells expressing the Selection Vector with random 6-amino acid library (X₆) fused to the carboxyl terminus of BLIP by a flexible linker are plated on non-permissive ampicillin to select for peptide masks which prevent unassisted inhibition of wildtype β-lactamase by BLIP. Selected masks are then tested in the Validation Vector for their ability to support reactivation of BLIP by docking to β-lactamase via fos-jun helix interaction. Finally, selected masks are tested for their ability to support activation by disinhibition of β-lactamase by transforming cells expressing the Validation Vector with the jun-thioredoxin disinhibitor vector.

### Definitions

A “binding pair member” refers to a molecule that participates in a specific binding interaction with a binding partner, which can also be referred to as a “second binding pair member” or “cognate binding partner”. Binding pairs include antibodies/antigens, receptor/ligands, biotin/avidin, and interacting protein domains such as leucine zippers and the like. A binding pair member as used herein can be a binding domain, i.e., a subsequence of a protein that binds specifically to a binding partner.

A “reference binding pair member” is a known binding pair member for which the practitioner wants to obtain a higher affinity binding analog i.e., an “improved” binding pair member.

An “affinity matured” or “improved” binding pair member is one that binds to the same site as an initial reference binding pair member, but has a higher affinity for that site.

Binding affinity is generally expressed in terms of equilibrium association or dissociation constants (Kₐ or K_{d}, respectively), which are in turn reciprocal ratios of dissociation and association rate constants (k_{d} and kₐ, respectively). Thus, equivalent affinities may correspond to different rate constants, so long as the ratio of the rate constants remains the same.

The terms “target molecule” or “”target target molecule” or “interaction inhibitor” are used interchangeably to refer to a molecule which interferes with the specific binding interaction between members of a binding pair. Typically a “target molecule” binds to one member of the binding pair, and thereby either directly or allosterically interferes with binding of the other binding pair member. The target molecule can be any number of molecules including peptides, chemicals, carbohydrates, lipids, etc.

The term “interaction” or “interacts” when referring to the interaction of binding pair members generally refers to specific binding to one another. However, it may also refer to indirect interaction mediated by other molecules, usually one other molecule. Accordingly, a molecule that interferes with the binding interaction of the binding pair members with one another decreases or prevents binding of a binding pair member to its binding partner. Typical binding pairs include antibodies/antigens, receptor/ligands, subunits of multimeric proteins or supra-molecular structures. “Binding” or “interacting” as used herein refers to noncovalent associations, e.g., hydrogen bonding, ionic bonding, electrostatic bonding, hydrophobic interaction, Van der Waals associations, and the like.

Binding of molecules will depend upon factors in solution such as pH, ionic strength, concentration of components of the assay, and temperature. In the binding systems described herein, the binding affinity of the binding pair members should be sufficient to permit interaction of the inhibitor and the reporter molecule, thus inactivating the reporter molecule when the binding pair members interact. Preferably, dissociation constants of binding pairs should be less than working concentrations, often about one-tenth, but generally not greater. Non-limiting examples of dissociation constants of the binding pair members in a solution, such as a in a cell interior, are typically 1 μM or less and preferably about 0.1 μM.

“Docking” refers to a binding interaction between any two molecules such as an antigen and antibody, a reporter and inhibitor, a mask and an inhibitor and the like.

“Domain” refers to a unit of a protein or protein complex, comprising a polypeptide subsequence, a complete polypeptide sequence, or a plurality of polypeptide sequences where that unit has a defined function. The function is understood to be broadly defined and can be binding to a binding partner, catalytic activity or can have a stabilizing effect on the structure of the protein. “Domain” also refers to a structural unit of a protein or protein complex, comprising one or more polypeptide sequences where that unit has a defined structure which is recognizable within the larger structure of the native protein. The domain structure is understood to be semi-autonomous in that it may be capable of forming autonomously and remaining stable outside the context of the native protein.

A “member” or “component” of a reporter system refers to a reporter molecule, a fragment or subsequence of a reporter molecule, a subunit of a reporter molecule, or an activator or inhibitor of the reporter molecule. The reporter molecule can be a complete polypeptide, or a fragment or subsequence thereof that retains reporter activity.

“Link” or “join” or “fuse” refers to any method of functionally connecting peptides, typically covalently, including, without limitation, recombinant fusion of the coding sequences, covalent bonding, and disulfide bonding. In the systems of the invention, a binding pair member is typically linked or joined or fused, often using recombinant techniques, at the N-terminus or C-terminus by a peptide bond to a reporter molecule or to an activator or inhibitor of the reporter molecule. However, the binding pair member may also be inserted into the reporter or inhibitor at an internal location that can accept such insertions.

The binding pair member can either directly adjoin the fragment to which it is linked or fused, or it can be indirectly linked or fused, e.g., via a linker sequence.

“Heterologous”, when used with reference to portions of a protein, indicates that the protein comprises two or more domains that are not found in the same relationship to each other in nature. Such a protein, e.g., a fusion protein or a conjugate protein, contains two or more domains from unrelated proteins arranged to make a new functional protein. Heterologous may also refer to a natural protein when it is found or expressed in an unnatural location such as when a mammalian protein is expressed in a bacterial cell.

A “low-affinity inhibitor” is a relative term referring to an inhibitor of the reporter molecule that has a K_{d} (equilibrium dissociation constant) for the reporter which is at least ten-fold higher than the working concentration of the inhibitor, such that the inhibitor cannot bind to the reporter to an appreciable extent without a heterologous mechanism for bringing the two together. For example, when under working conditions in vitro or in vivo the inhibitor concentration is ten-fold lower than its K_{d} for the reporter, the reporter will be only ˜10% inhibited. However, if each is linked to a different member of a binding pair, and the K_{d} of the binding pair is at least 10-fold lower than the working concentration of the inhibitor fusion, then the reporter will be more than 90% inhibited. Therefore, the optimal concentration of the inhibitor fused to a binding pair member is at least 10-fold below the inhibitor K_{d} and at least 10-fold above the binding pair K_{d}. The optimal concentration of the reporter fused to a binding pair member is equivalent to or slightly below that of the inhibitor fusion.

“Mask” refers to a molecule that has low affinity for a reporter or inhibitor, such that the mask does not bind appreciably at working concentrations unless it is tethered covalently to the reporter or inhibitor. Further, binding of the mask to the inhibitor prevents the inhibitor from binding to the reporter and vice versa. It should be noted that in this system, a reporter mask inhibits binding of the inhibitor, but does not inhibit reporter activity. In other systems, reporter masks may be used which inhibit reporter activity. A mask allows a high-affinity inhibitor to be used without fear of increasing the background inhibition because its association rate constant is greatly reduced without affecting the dissociation rate constant of the reporter-inhibitor complex, thereby reducing the overall affinity while retaining the stability of the high-affinity reporter-inhibitor complex. This has the advantage of allowing the binding pair interaction to operate like a switch. This switch property renders the system much more robust with respect to the steric constraints which may be imposed by the binding pair interaction on inhibitor binding.

A “tripartite” molecule refers to a conjugate molecule comprising three components: 1.) a binding pair member, 2.) an inhibitor or a reporter, and 3.) a mask. The three components can be linked in any order.

A “competitor” is any molecule that competes with a test binding pair member for binding to the cognate binding partner. A “competitor” can also refer to a binding pair member that competes with a target molecule for binding to the cognate binding partner. Often, the competitor is fused to the inhibitor. However, in other embodiments, the competitor may be fused to the reporter, for example, when the K_{d} of the competitor for the other binding pair member is substantially lower than their working concentrations, but their working concentrations are similar.

“Antibody” refers to a polypeptide comprising at least a heavy chain variable region and a light chain variable region that together specifically bind and recognize an antigen, the variable regions being specified by immunoglobulin genes. Recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (VL) and variable heavy chain (VH) refer to these light and heavy chain variable regions respectively.

Antibodies exist, e.g., as intact immunoglobulins, as a number of well-characterized fragments produced by digestion with various peptidases, or as well-characterized fragments produced by recombinant gene expression. Thus, for example, pepsin digests an antibody below the disulfide linkages in the hinge region to produce F(ab)′2, a dimer of Fab which itself is a light chain joined to VH-CH1 (Fd fragment) by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defmed in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

As used herein, the term “single-chain antibody” (scFv) refers to a polypeptide comprising a VH domain and a VL domain in polypeptide linkage, generally linked via a spacer peptide (e.g., [Gly-Gly-Gly-Gly-Ser]ₓ), and which may comprise additional amino acid sequences at the amino- and/or carboxyl-termini. For example, a single-chain antibody may comprise a tether segment for linking to the encoding polynucleotide. As an example, a scFv is a single-chain antibody. Single-chain antibodies are generally proteins consisting of one or more polypeptide segments of at least 10 contiguous amino acids substantially encoded by genes of the immunoglobulin superfamily (e.g., see The Immunoglobulin Gene Superfamily, A. F. Williams and A. N. Barclay, in Immunoglobulin Genes, T. Honjo, F. W. Alt, and T. H. Rabbitts, eds., (1989) Academic Press: San Diego, Calif., pp.361-387, which is incorporated herein by reference), most frequently encoded by a rodent, non-human primate, avian, porcine, bovine, ovine, goat, or human heavy chain or light chain gene sequence. A functional single-chain antibody generally contains a sufficient portion of an immunoglobulin superfamily gene product so as to retain the property of binding to a specific target molecule, typically a receptor or antigen (epitope).

As used herein “antibody” may also refer to any functional, i.e., capable of binding specifically to an epitope, VH and VL pair that are each linked in various configurations to other polypeptide(s) that may perform various functions, e.g.,as reporter, reporter inhibitor, or stabilizer of the VH-VL complex.

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al, Biotechnology 10:779-783 (1992)).

As used herein “immunoglobulin variable region domain” refers to any VH or VL domain used as a binding moiety without a companion VH or VL domain. As with antibodies, such domains may be linked in various configurations to other polypeptide(s) that may perform various functions, e.g., as reporter, reporter inhibitor, or reporter activator.

As used herein “ligand” refers to a molecule that is recognized by, i.e., binds to, a particular receptor. As one of skill in the art will recognize, a molecule (or macromolecular complex) can be both a receptor and a ligand, typically when both are soluble or both are membrane-bound. However, when one is membrane-bound and the other is soluble, the former is commonly referred to as the receptor and the latter is the ligand. When both are soluble, the binding partner having a smaller molecular weight is typically referred to as the ligand and the binding partner having a greater molecular weight is referred to as a receptor. More generally, the binding partners of non-receptor proteins may also be referred to as ligands.

A “linker” or “spacer” refers to a molecule or group of molecules that covalently connects two molecules, such as a binding pair member and a reporter molecule or an inhibitor, and serves to place the two molecules in a preferred configuration, e.g., so that a reporter molecule can interact with an activator or inhibitor with minimal steric hindrance from a binding pair member, and a binding pair member can bind to a binding partner with minimal steric hindrance from the reporter or inhibitor.

The term “flexible linker” refers to a peptide linker of any length whose amino acid composition is rich in glycine to minimize the formation of rigid structure by interaction of amino acid side chains with each other or with the polypeptide backbone. A typical flexible linker would have the composition (Gly₄Ser)ₓ.

The term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame.

The term “expressing components of a selection system” refers to culturing a cell population under conditions in which nucleic acid sequences comprised by expression vectors encoding members of a selection system are expressed.

A “scaffolded peptide” refers to a peptide, typically of up to about 20 amino acids in length, that is inserted into a natural protein at a location known to accept such insertions without interfering with the folding or native configuration of the protein (A Skerra, Engineered protein scaffolds for molecular recognition. J Mol Recognit 2000 July-August; 13(4):167-87). Usually the location is on the surface of the protein. Often, the peptide is not a known natural sequence, and therefore is not expected to fold into a stable structure on its own, but generally assumes a random coil structure in solution. However, when inserted into the scaffold protein the peptide is expected to acquire some degree of stable structure by packing against the surface of the protein. Such structure generally improves the ability of the peptide to bind with high affinity to other molecules, such as other proteins. Many proteins may serve as scaffolds for random peptide libraries. Frequently, surface loops between elements of secondary structure such as α-helixes or strands of a β-sheet may accept such insertions without significant perturbation of folding or structure. Examples of proteins that have been used as scaffolds include, but are not limited to, thioredoxin (or other thioredoxin-like proteins), nucleases (e.g., RNase A), proteases (e.g., trypsin), protease inhibitors (e.g., bovine pancreatic trypsin inhibitor), antibodies or structurally-rigid fragments thereof, and other domains of the immunoglobulin superfamily.

The term “library of expressed sequences” refers to any population of nucleotide sequences which are derived from messenger RNA, and which are therefore understood to encode polypeptide sequences which are produced naturally in cells.

Each of the above terms is meant to encompass all that is described, unless the context dictates otherwise.

### DETAILED DESCRIPTION OF THE INVENTION

### Introduction

The target-activatable reporter systems of the current invention overcome limitations of prior art reporter fragment complementation systems such as fragment instability and low specific activity. Further, the current invention fills an unmet need for systems capable of positive selection of inhibitors of molecular interactions of interest. The invention provides a system that uses stable components, e.g., an intact reporter molecule such as a native enzyme or a stable catalytic domain of the enzyme, as the target-activated form. This is accomplished by engineering low-affinity inhibitors and activators of the reporter molecule, the activators typically being inhibitors of the inhibitors.

Both the inhibitors and activators exert their effects by being “docked” to the reporter molecule either through a heterologous interaction or by direct polypeptide linkage. A number of configurations of the target-mediated disinhibition system for reporter activation are possible.

By way of example, systems that use intact wild-type or mutant β-lactamases with natural or engineered β-lactamase inhibitor proteins (e.g., BLIP; Strynadka et al., 1996, Nature Struct. Biol. 3:290-297), and catalytically inactive mutants of β-lactamase in different combinations are described. They can be used for many applications, for example to select binding pair members with increased affinity by affinity competition in bacterial cells.

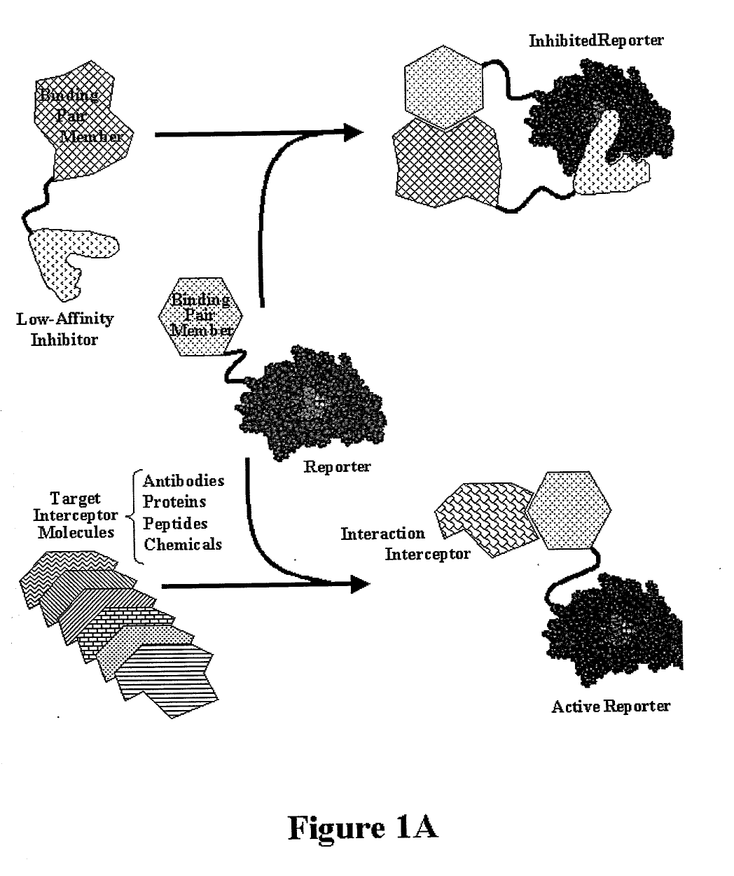

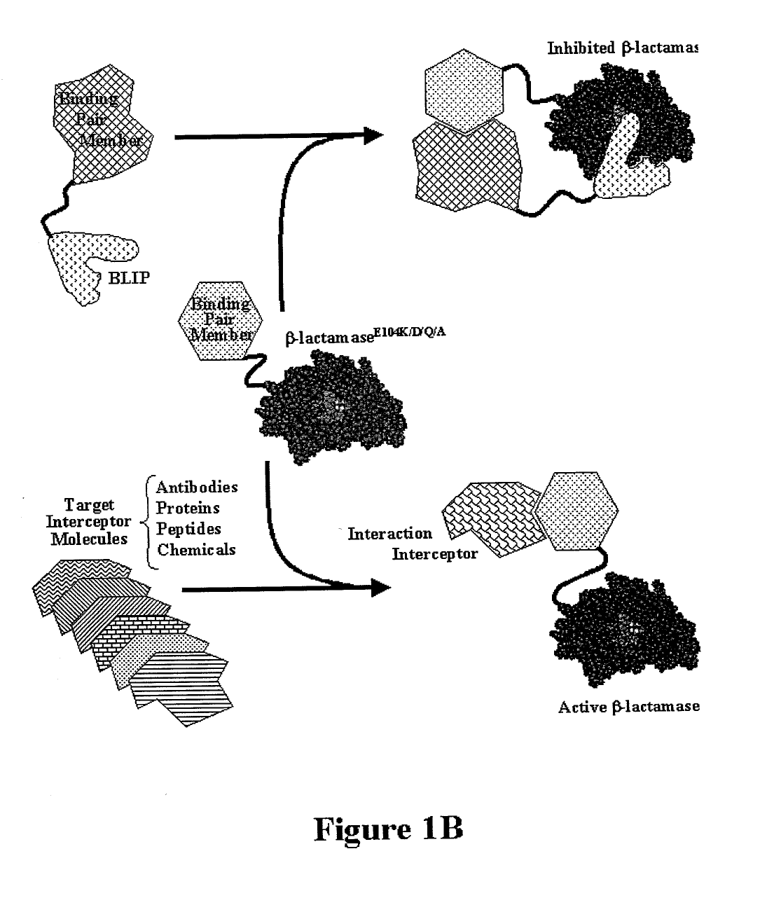

In one embodiment, the components of the system comprise mutants of the enzyme β-lactamase and a β-lactamase inhibitor protein (BLIP). The β-lactamase mutants Glu104Lys/Gln/Ala/Asp all have reduced affinities for BLIP but near-wild-type enzymatic activities. When expressed at appropriate levels in cells, or when used at appropriate concentrations in vitro, these mutants are not appreciably inhibited by BLIP. However, when the mutant enzymes and inhibitor are fused to heterologous proteins, i.e., binding pair members, and when the binding pair members interact with one another, the inhibitor is docked to the enzyme and the enzyme becomes inactivated (see FIGS. 1A and 1B). This provides for activation of the enzyme by a target molecule that interferes with the binding interaction between the binding pair members. This system can be used, for example, for the positive selection of inhibitors of target interactions from chemical, antibody, or peptide libraries. Affinity maturation of antibodies can also be accomplished with such a system as a particular embodiment of antibody inhibitor selection.

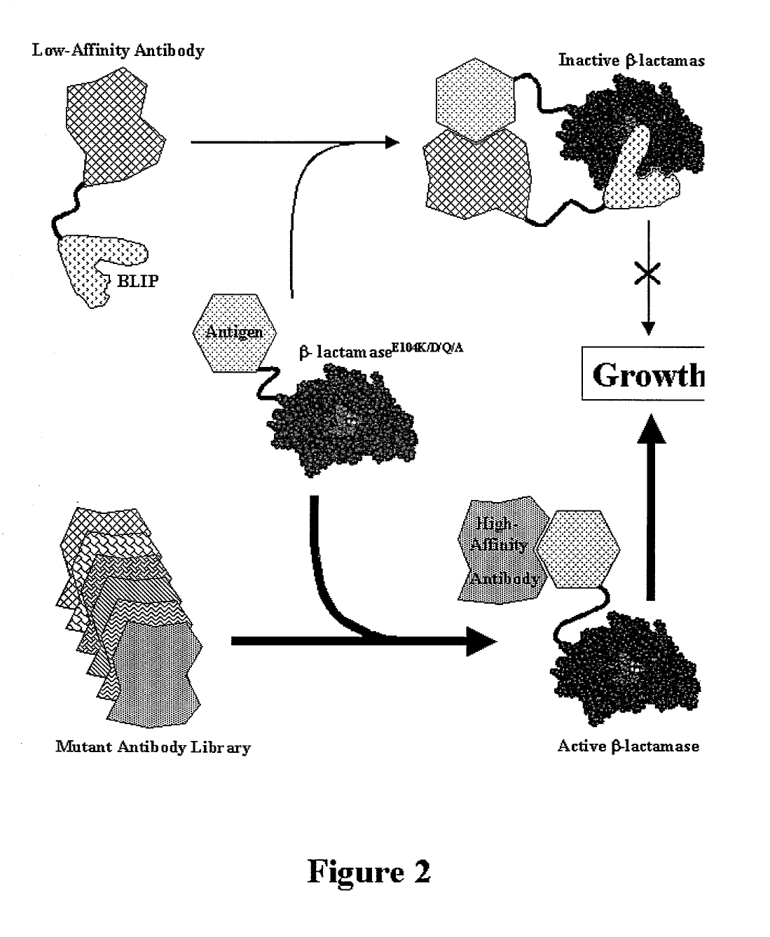

An example of the use of this system for affinity maturation is further illustrated in FIG. 2 (see FIG. 2). In this application of the system, a first member of a binding pair, a cognate binding partner, typically an antigen, is linked to a β-lactamase^{E104K} reporter (which was generated based on the x-ray structure of the BLIP/β-lactamase complex (Strynadka et al., Nat. Struct. Biol. 3:290-297, 1996)), and the other member, e.g., a reference binding pair member or a competitor molecule with the same affinity as the reference binding pair member, typically a low-affinity antibody, is linked to BLIP, such that when the binding pair members interact, BLIP is docked to β-lactamase^{E104K} and the latter is inactivated. In the presence of a library of test binding pair members, the β-lactamase^{E104K} reporter will become activated as test binding pair members bind to the cognate binding partner, thereby preventing the reference binding pair member from docking BLIP to the reporter. The activity of the β-lactamase_{E104K} reporter will be proportional to the affinity of the test binding pair member for the cognate binding partner, such that higher-affinity test binding pair members may be isolated by plating on solid medium containing β-lactam antibiotic concentrations which are non-permissive for the reference binding pair member. Additional increments in affinity may be obtained by subjecting a selected higher-affinity test binding pair member to a low level of random or site-specific mutagenesis, substituting the resultant mutagenic library as the test binding pair member library, and using the same higher-affinity test binding pair member as the new competitor.

In another embodiment of the interaction-inhibitor selection system, a mask is included as a component of the system. A mask is a molecule that has low affinity for the inhibitor or reporter, such that the mask does not bind appreciably at working concentrations inside the cell, unless it is tethered covalently to the inhibitor or reporter. Further, binding of the mask to the reporter or inhibitor prevents the one from binding to the other. In this system, reporter masks inhibit only inhibitor binding, not reporter activity. Modification of the selection system by introduction of a mask improves the dynamic range and control of the system by increasing the dependence of reporter-inhibitor complex formation on docking by the binding pair, while also increasing the stability of the reporter-inhibitor complex, thereby reducing the background activity of the inhibited reporter.

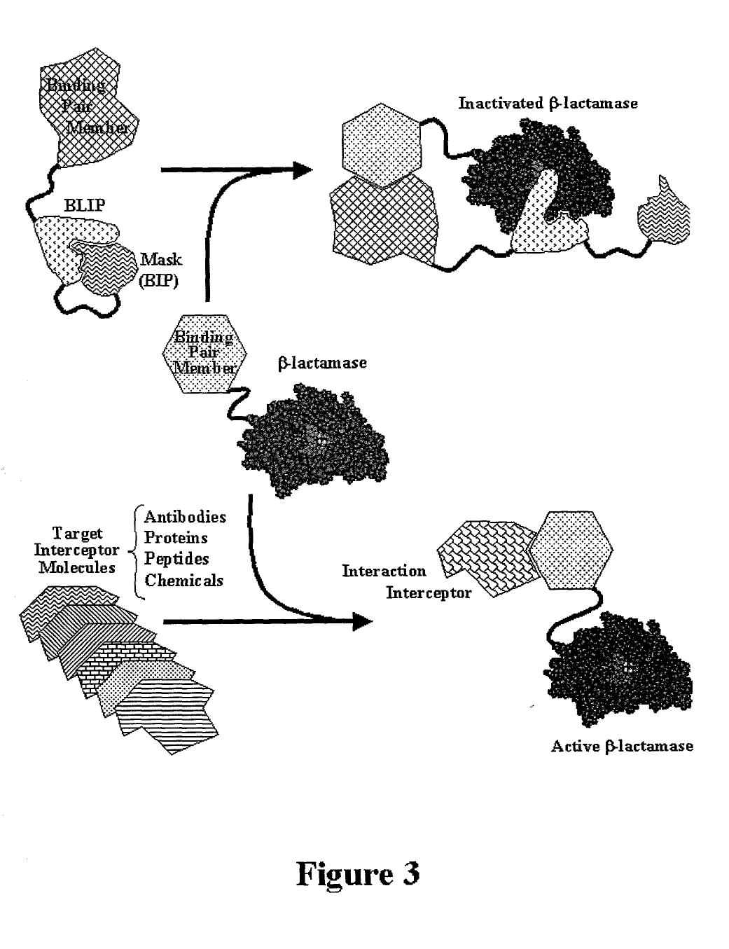

In one embodiment of the interaction-inhibitor selection system, one binding pair member is linked to the reporter and the other is linked to the inhibitor of the reporter. The mask modification involves fusing a mask to the inhibitor fusion component or to the reporter fusion component of the system, so that the masked component is now a tripartite fusion of one binding pair member to both the reporter or inhibitor and the mask. In the system dipicted in FIG. 3, the reporter inhibitor is constitutively bound to the mask until it is docked to the reporter by the interaction of the binding pair, whereupon the higher affinity of the inhibitor for the reporter than for the mask causes it to shift from the mask to the reporter, thereby inactivating the reporter. The mask modification with the high-affinity reporter-inhibitor complex alters the binding kinetics of the interaction-inhibitor selection system by decreasing both the association and dissociation rate constants, such that the K_{d} remains high, i.e., the overall affinity remains low, but the reporter-inhibitor complex is stabilized. This has the advantage of allowing the binding pair interaction to operate like a switch so long as the rates of reporter-inhibitor association and dissociation are both low compared to the rate of protein accumulation. This switch property renders the system much more robust with respect to the steric constraints which may be imposed by the binding pair interaction on inhibitor binding. Also, the dynamic range of this system, i.e., the scalar difference between the reporter activity due to an inhibitor of the binding pair interaction and the residual reporter activity in the absence of such an inhibitor, is increased.

The systems of the current invention thus provide methods of detecting the presence of a target molecule in a biological sample. Such a target molecule may act competitively and/or allosterically to activate a reporter molecule. As described above, one member of a binding pair is linked to the reporter molecule and the second member of the binding pair is linked to a low-affinity inhibitor of the reporter molecule. A compound that interferes with the binding interaction between the binding pair members can then activate the reporter molecule. Accordingly, target molecules can be identified by detection of the activated reporter. It is to be appreciated that in this embodiment either member of the binding pair can be joined to the reporter molecule as long as the other member of the binding pair is joined to the inhibitor.

The binding pair members are typically proteins, such as an antibody and its cognate antigen. Such a system can be used for a number of applications. For example, antibodies with a higher affinity for the antigen than a first, reference antibody can be identified (see FIG. 2). Such a procedure can be performed, for example, by co-expressing the reporter and inhibitor fused to the antigen and reference antibody with a library of mutants of the antibody, and screening for reporter activity that is higher than that produced by co-expression of the reporter and inhibitor fusion proteins with the unmutated antibody.

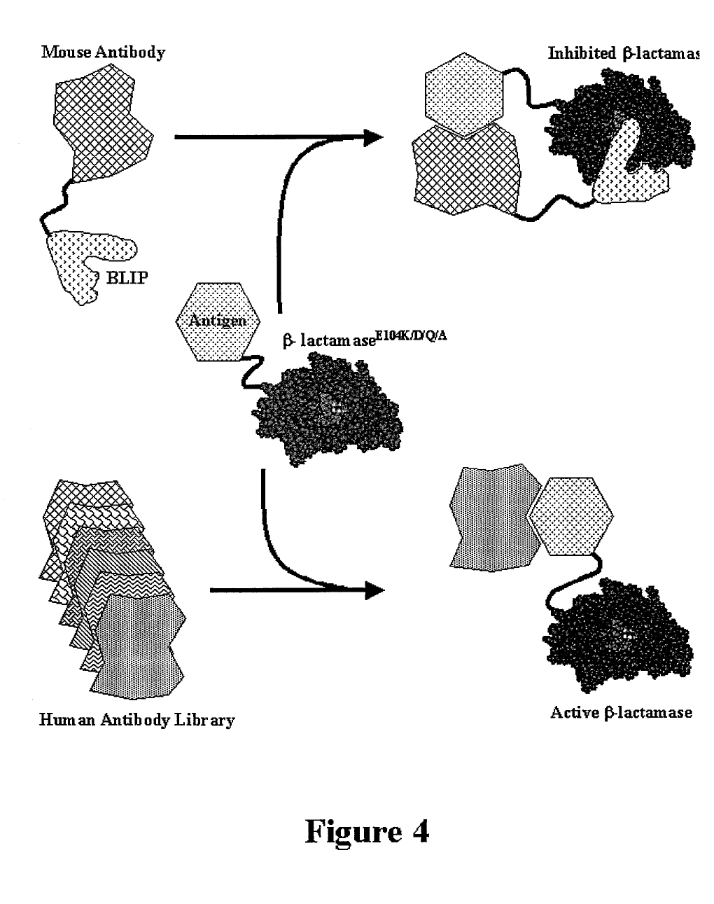

In other applications, antibodies with other properties can be selected. For example, if the parent antibody is from a mouse, than human antibodies for the same epitope on the same antigen could be selected by co-expressing the fusion proteins with a human antibody library and selecting for activation of the enzyme (see FIG. 4).

The system can also be used to screen for inhibitors of any target interaction by co-expressing or exposing the fusion proteins either in cells or in vitro with candidate inhibitors and selecting for activation of the reporter. The candidates can be screened either individually or collectively.

For protein binding pair members and reporters/inhibitors comprised by the conjugate molecules of the invention, the protein sequence included in the conjugate or fusion protein can encode all of the protein or a fragment of the protein. The protein binding pair members are often genetically fused, either at the N-terminus or C-terminus, to the reporter, or inhibitor or activator. Often, fusion is through a linker. Peptide or scaffolded peptides can also be incorporated into the fusion molecules. As appreciated by one of skill in the art, the binding pair members can bind either directly or via additional molecules, which my be produced by cells into which the fusion molecules are introduced or added to growth medium.

### Binding Pair Members

Any number of binding pairs are useful in practicing the invention. These include antibody/antigen binding partners, receptor/ligand binding partners, interacting subunits of enzymes, and proteins that interact in intra-cellular signal transduction, gene regulation, such as transcription factors, and regulation of metabolism. Members of the latter category include a number of transcription factors, for example, c-fos and c-jun.

Binding partners that involve a member that is not a protein can also be used. Thus, a binding pair member can be, e.g., a small molecule, a carbohydrate, a lipid, or nucleic acid, as well as portions, polymers and analogues thereof, provided they are capable of being linked to the reporter or inhibitor. For example, small molecule binders may be used by conjugating them to a chemical tag such as biotin. Such conjugates typically can diffuse freely into the bacterial periplasm, allowing them to serve as cognate binding partners, e.g., to screen for higher affinity test binding partners. For example, a binding pair member that binds to a small molecule binding partner can be linked to a reporter molecule and an inhibitor can be linked to a protein that binds to the tag, such as avidin or streptavidin for a biotin tag. When the binding pair member binds to the small molecule cognate binding partner, and the linked tag binds to the tag-binder, the reporter and inhibitor are brought into proximity and the reporter is inactivated. In the presence of a target molecule that interferes with the binding interaction, the reporter molecule is activated. In selecting a target molecule that has a higher affinity for the small molecule binding partner than that of the binding pair member, the resulting reporter activity, and dependent phenotype, will be proportional to the affinity of the target molecule, thereby providing the basis for selection of higher-affinity binders of small molecules of interest.

Examples of small molecule binding pair members include steroids, sterols and related molecules that bind to steroid hormone receptors; prostaglandins and related molecules that bind to prostaglandin receptors; porphyrins and relatives such as hemes and Vitamin B12 that bind as co-factors to enzymes and electron transport proteins; biogenic amines such as the catecholamine neurotransmitters and their receptors; other vitamins and nutrients for which uptake receptors are present on cells; ATP, GTP, cAMP, and cGMP, all of which bind to many proteins whose activities are regulated thereby, such as G proteins, G protein coupled receptors, cytoskeletal proteins, transcription factors, chaperones, etc.”

Further examples of binding pair members are found in U.S. Pat. Nos. 6,294,330; 6,220964; 6,342,345; and/or U.S. patent application Ser. No. 09/526,106, filed on Mar. 15, 2000, which are hereby incorporated by reference.

### Target Molecules

The methods of the invention can be used to detect any number of target molecules. A target molecule binds to one member of a binding pair, which is fused to either the reporter or the inhibitor, but preferably the former, thereby preventing the binding of the second member of the binding pair, and thereby preventing docking of the inhibitor to the reporter. In some embodiments, binding of the target molecule may cause an allosteric change to displace or prevent binding of the second member of the binding pair. A target molecule can also competitively prevent binding of the second binding pair member.

The target molecule can be any number of molecules. These include proteins, peptides, lipids, carbohydrates, chemical compounds, and the like. For example, a target molecule can be an antibody that binds to an epitope (on a binding pair member) that is fused to the reporter molecule. The target antibody can then compete with the binding of the partner binding pair member to the epitope. Such a partner binding pair member can, for example, also be an antibody to the epitope. Thus, a target antibody such as an antibody of higher affinity that the partner antibody, will activate the reporter molecule by blocking the binding of the antibody binding partner to the epitope, thereby preventing binding of the inhibitor to the reporter molecule. As appreciated by those of skill in the art, the epitope can be a scaffolded peptide or other artificial binding proteins, natural ligands, or antibodies that bind to discreet loci on the antigen surface.

### Reporter Molecules

A variety of different reporter molecules can be used in the systems and methods of the invention. Typically, the reporter molecules are enzymes. Examples of such enzymes can be found in WO 00/71702. These include antibiotic resistance markers such as β-lactamase, penicillin-amidases, aminoglycoside phosphotransferases, e.g., neomycin phosphotransferase, puromycin N-acetyltransferase (Sanchez-Puig et al., Gene 257:57-65, 2000), and chloramphenicol acetyl transferase. For example, β-lactamase is often used as a reporter molecule. The enzyme has a k_{cat} in the range of 10⁴ sec⁻¹ for some antibiotic substrates, e.g., ampicillin, and with such activity it can be estimated that as little as ten molecules of the activated intact enzyme per bacterial cell is sufficient to allow a single cell to grow into a colony overnight on solid medium containing a lethal concentration of the antibiotic.

Other enzyme reporter molecules that provide a selectable phenotype can also be used. These include enzymes that can hydrolyze chromogenic or fluorogenic substrates to yield a colored or fluorescent product. Such enzymes include β-galactosidase, alkaline phosphatase, peroxidases, esterases, carboxypeptidases, glycosidases, glucuronidases, and carbamoylases.

Non-enzymatic reporter molecules can also be employed using the methods of the invention. For example, Green Fluorescent Protein (GFP) of Aequorea Victoria (Chalfie et al., (1994) Science 263: 802-805) can be employed as a reporter. GFP absorbs blue light and fluoresces green. An inhibitor of GFP would quench fluorescence when brought into proximity of the GFP by the binding pair members. A molecule that blocks interaction of the binding pair would prevent docking of the inhibitor to GFP, thereby allowing GFP to fluoresce, thus providing a detectable signal. An inhibitor of GFP suitable for target-mediated disinhibition could be isolated by any of various methods. For example, molecules which bind to GFP could be isolated from antibody, immunoglobulin variable region, or scaffolded peptide libraries by phage display methods (Phage Display of Pegtides and Proteins Kay, Winter, and McCafferty, Eds. (1997) Academic Press, San Diego), or by β-lactamase fragment complementation (U.S. patent application Ser. No. 09/526,106).

Further examples of reporter molecules are found in U.S. Pat. Nos. 6,294,330; 6,220964; 6,342,345; and/or U.S. patent application Ser. No. 09/526,106, filed on Mar. 15, 2000, which are hereby incorporated by reference.

### Inhibitor Molecules

Inhibitor molecules of use in the invention are those molecules that inhibit the activity of the reporter molecules. The inhibitors have an affinity for the reporter which corresponds to a K_{d} that is at least ten-fold higher than the concentrations at which the inhibitor is typically used.

For example, a low-affinity enzyme inhibitor should have a K_{I} for the enzyme that is typically 10-100-fold higher than the optimal intracellular concentration of the inhibitor, so that the enzyme is 90%-99% active in the absence of an interaction, assuming that the enzyme is completely inhibited when the inhibitor is bound, in which case the K_{d} and Kᵢ are roughly equivalent.

In some cases, such as in growing cells, where the system may operate far from equilibrium, “low affinity” may refer specifically to low association rate, defined as less than one-tenth of the rate of protein accumulation in the system, so that in the absence of a docking interaction with a high association rate (i.e., greater than the rate of protein accumulation), the reporter will remain >90% active.

Examples of natural inhibitors of enzyme reporters include the β-lactamase inhibitor protein (BLIP; Strynadka et al. (1994) Nature 368: 657-660). BLIP is a 165 amino acid protein that is a natural inhibitor of TEM-1, a variant of β-lactamase and has a Kᵢ for β-lactamase in the range 0.1-1.0 nM. Natural protein inhibitors also exist for many other enzymes.

Low-affinity protein inhibitors for any reporter protein can also be engineered from other proteins. For example, this can be performed using a scaffolded random peptide library. The reporter protein of interest, which is used to select the inhibitor, and the scaffolded peptide library can be used in any of a variety of systems that detect protein-protein interactions, such as bacteriophage display (Phage Display of Peptides and Proteins Kay, Winter, and McCafferty, Eds. (1997) Academic Press, San Diego) or β-lactamase fragment complementation β-lactamase fragment complementation (U.S. patent application Ser. No. 09/526,106). Further, a number of different proteins may be used as scaffolds. For example, thioredoxin has been widely used. Peptide libraries, typically of 6-20 random amino acids, can be inserted into the active site of thioredoxin without disturbing its stability. Thioredoxin has the further advantage that it is much smaller than most natural inhibitors, and is therefore less sterically constrained when access to the reporter is restricted by linker lengths and binding pair orientation. For example, BLIP is ˜19 kDa in size, whereas thioredoxin is only ˜11 kDa. Another good scaffold is the immunoglobulin domain, of which the antibody variable region domain is a prime example (Skerra (2000) J Mol Recognit 13:167-87). The immunoglobulin superfamily is one of the largest families of structurally homologous protein folds found in nature (Hawke et al. (1999) Immunogenetics 50:124-33). Immunoglobulin domains are comparable in size to thioredoxin and tolerate random peptide libraries in a number of exposed loops in the structure.

To select a reporter inhibitor, peptide libraries, e.g., a thioredoxin-scaffolded peptide (trxpep) library can be displayed, e.g., on the surface of filamentous bacteriophage, and panned against the immobilized reporter. Phage which bind to the reporter can then be recovered, and the encoded trxpeps can be individually screened for their ability to inhibit the reporter only when both are fused to cognate binding pair members. It is reasonable to expect that a substantial proportion of reporter-binding trxpeps will also inhibit the function of the reporter. In many cases, the reporter itself may be used to screen trxpep libraries for low-affinity inhibitors. The only requirement is that a null reporter phenotype be selectable. For example, the reporter and trxpep library can be fused to each member of a model binding pair, such as the leucine zipper helices from the fos and jun transcription factors, and expressed in cells. If the reporter is fluorescent, or produces a colored or fluorescent product, an inhibitor trxpep will, upon docking to the reporter by the binding pair interaction, render the host cells colorless or non-fluorescent, and this can be detected by eye or by flow cytometry.

Other scaffold proteins can also be used as a reagent to select reporter inhibitors (or masks, further described below). These proteins are typically small in size (e.g., less about 200 amino acids), rigid in structure, of known three dimensional configuration, and are able to accommodate insertions of peptides of interest without undue disruption of their structures. An important feature of such proteins is the availability, on their solvent exposed surfaces, of locations where peptide insertions can be made (e.g., the thioredoxin active-site loop). Typically such scaffold proteins can be expressed at high levels in various prokaryotic and eukaryotic hosts, or in suitable cell-free systems. Furthermore, the scaffold proteins are generally soluble and resistant to protease degradation. Examples of additional scaffold proteins useful in the invention include RNase A, proteases (e.g., trypsin), protease inhibitors (e.g., bovine pancreatic trypsin inhibitor), antibodies or fragments thereof, and immunoglobulins.

### Mask Molecules

Mask molecules can also be engineered from natural proteins or other molecules in a variety of ways. For example, an inhibitor mask for the BLIP inhibitor of β-lactamase can be generated from β-lactamase itself. The active site nucleophile can, for 30 instance, be changed to eliminate enzymatic activity. Furthermore, the affinity for BLIP can be reduced by mutating specific residues. When such a molecule is fused to a fusion of a binding pair member to BLIP, the latter will be constitutively inactive. When it is “docked” to β-lactamase by the interaction of the binding pair members, the higher affinity of BLIP for β-lactamase will cause BLIP to transfer from the mask to the β-lactamase, thereby inactivating the enzyme. A target molecule that interferes with the binding pair interaction will then prevent the transfer of BLIP to the β-lactamase and thus result in activation of the enzyme.

An exemplary system using a β-lactamase and the BLIP inhibitor with a mask can be generated as follows and is illustrated in FIG. 3. The components to which the binding pair members are linked comprise β-lactamase and BLIP fused to BIP (BLIP Inhibitor Protein). BIP is a catalytically-inactive β-lactamase mutant in which the active site nucleophile, Ser70, has been replaced by Ala (S70A). To allow its use as a mask for BLIP a further mutation has been introduced into BIP (Glu104Lys/Gln/Asp/Ala, E104K/Q/D/A) to reduce its affinity for BLIP. Whereas, under preferred conditions of gene expression or concentration, β-lactamase is constitutively inhibited by free BLIP, such is not the case when BLIP is fused to this low-affinity BIP mutant. However, when brought into similar proximity of β-lactamase by the interaction of binding pair members, the 100-fold higher affinity for BLIP of the wild-type β-lactamase allows the latter to displace BIP from BLIP, thereby inhibiting β-lactamase.

In the presence of a molecule that prevents the binding interaction between the binding pair members, presumably by binding to one or the other binding pair member, the BLIP-BIP fusion is not docked to β-lactamase, and the latter remains active . β-lactamase activity is therefore increased relative to its activity in the absence of the competing molecule, typically by an amount which is inversely proportional to the K_{d} of the binding pair interaction. This means that such a system could be used for affinity maturation of antibodies or other binding molecules by virtue of its ability to identify higher-affinity variants thereof in libraries of test binding pair members comprised of the subject binding molecule subjected to random mutagenesis (See FIGS. 2 and 3). For example, test binding pair members with higher affinities for the cognate binding partner than a reference binding pair member, e.g., the parent binding molecule, will produce higher β-lactamase activities, and may therefore be isolated by growth on solid medium containing β-lactam antibiotic concentrations which are non-permissive for the reference binding pair member. Additional increments in affinity may be obtained by subjecting a selected higher-affinity variant to a low level of random mutagenesis, substituting the resultant mutagenic library as the test binding pair member library, and using the same higher-affinity variant as the new competitor. Accordingly, such a system can be used for affinity maturation (see, e.g., co-pending U.S. patent application filed oct. 31, 2001, Affinity Maturation by Competitive Selection; Balint, Her and Larrick, Inventors).

Low-affinity inhibitor masks suitable for the same applications can also be selected from libraries of random peptides, scaffolded random peptides, or other binding proteins with binding diversity such as immunoglobulin variable regions in a method analogous to that described for selecting inhibitors. For example, a mask for BLIP could easily be selected from a peptide library by fusing the peptide library to BLIP, and co-expressing this fusion in bacteria with β-lactamase under conditions in which the β-lactamase is strongly inhibited. Masks are then selected simply by plating on restrictive ampicillin. Selected masks are then screened for the ability to permit docked reactivation of the masked molecule. This is accomplished by expressing β-lactamase and the BLIP-mask fusions as fusions to model binding pair members, and testing for restoration of β-lactamase activity. In a convenient embodiment, the model binding pair is comprised of two binding molecules which bind to non-overlapping epitopes on the same antigen, and the free antigen is co-expressed in the system from an inducible promoter. In this way the masks may be selected with the antigen gene turned off, so that the binding pair interaction does not occur, and, when successfully masked, BLIP cannot inhibit β-lactamase. Selected masks can then be screened for extinction of β-lactamase activity when the antigen gene is turned on. This will allow the binding pair interaction to occur, thereby docking the masked BLIP to β-lactamase, whereupon the higher affinity of the latter for BLIP will allow it to displace the mask, and β-lactamase will become inactivated.

Low-affinity masks for the reporter that are suitable for the same applications can also be selected from libraries of random peptides, scaffolded random peptides, or other binding proteins with binding diversity such as immunoglobulin variable regions. This is accomplished simply by co-expressing a high-affinity inhibitor with the reporter fused at either terminus to a random peptide library via a flexible linker, and selecting for reporter activity under expression conditions in which inhibition of the reporter would normally be constitutive. For example, BLIP would normally inhibit wild-type β-lactamase constitutively, but a peptide mask could be selected which would protect β-lactamase from BLIP without inhibiting β-lactamase itself. As for the BLIP masks, the selected β-lactamase masks can then be tested in the same way for their ability to support re-inhibition of the enzyme upon docking to BLIP by the interaction of a model binding pair.

### Generation of Conjugate Molecules

The reporter and inhibitor conjugates can be joined by methods well known to those of skill in the art. These methods include both chemical and recombinant means.

Chemical means of joining the heterologous domains are described, e.g., in Bioconjugate Techniques, Hermanson, Ed., Academic Press (1996). These include, for example, derivitization for the purpose of linking the moieties to each other, either directly or through a linking compound, by methods that are well known in the art of protein chemistry. For example, in one chemical conjugation embodiment, the means of linking the reporter molecule (or inhibitor) to the binding pair member comprises a heterobifuncitonal coupling reagent that ultimately contributes to formation of an intermolecular disulfide bond between the two moieties. Other types of coupling reagents that are useful in this capacity for the present invention are described, for example, in U.S. Pat. No. 4,545,985. Alternatively, an intermolecular disulfide bond can be formed between cysteine residues present in each of the protein molecules to be linked. The cysteines can occur naturally or are inserted by genetic engineering. The means of linking moieties may also use thioether linkages between heterobifunctional crosslinking reagents or specific low pH cleavable crosslinkers or specific protease cleavable linkers or other cleavable or noncleavable chemical linkages.

The means of linking the heterologous domains of the protein can also comprise a peptidyl bond formed between domains that are separately synthesized by standard peptide synthesis chemistry or recombinant means. The protein itself can also be produced using chemical methods to synthesize an amino acid sequence in whole or in part. For example, peptides can be synthesized by solid phase techniques, such as, e.g., the Merrifield solid phase synthesis method, in which amino acids are sequentially added to a growing chain of amino acids (see, Merrifield (1963) J. Am. Chem. Soc., 85:2149-2146). Equipment for automated synthesis of polypeptides is commercially available from suppliers such as PE Corp. (Foster City, Calif.), and may generally be operated according to the manufacturer's instructions. The synthesized peptides can then be cleaved from the resin, and purified, e.g., by preparative high performance liquid chromatography (see Creighton, Proteins Structures and Molecular Principles, 50-60 (1983)). The composition of the synthetic polypeptides or of subfragments of the polypeptide, may be confirmed by amino acid analysis or sequencing (e.g., the Edman degradation procedure; see Creighton, Proteins, Structures and Molecular Principles, pp. 34-49 (1983)).

In some embodiments, nonclassical amino acids or chemical amino acid analogs can be introduced as a substitution or addition into the sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxy-proline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

In another embodiment, the reporter and inhibitor conjugates, are joined via a linking group. The linking group can be a chemical crosslinking agent, including, for example, succinimidyl-(N-maleimidomethyl)-cyclohexane-1-carboxylate (SMCC). The linking group can also be an additional amino acid sequence(s), including, for example, a polyalanine, polyglycine or similar linking group.

In a specific embodiment, the coding sequences of each polypeptide in the fusion protein are directly joined at their amino- or carboxy-terminus via a peptide bond in any order. Alternatively, an amino acid linker sequence may be employed to separate the first and second polypeptide components by a distance sufficient to ensure that each polypeptide folds into its secondary and tertiary structures. Such an amino acid linker sequence is incorporated into the fusion protein using standard techniques well known in the art. Suitable peptide linker sequences may be chosen based on the following factors: (1) their ability to adopt a flexible extended conformnation; (2) their inability to adopt a secondary structure that could interact with finctional epitopes on the first and second polypeptides; and (3) the lack of hydrophobic or charged residues that might react with the polypeptide functional epitopes. Typical peptide linker sequences contain Gly, Val and Thr residues. Other near neutral amino acids, such as Ser and Ala can also be used in the linker sequence. Amino acid sequences which may be usefully employed as linkers include those disclosed in Maratea et al. (1985) Gene 40:39-46; Murphy et al. (1986) Proc. Natl. Acad. Sci. USA 83:8258-8262; U.S. Pat. Nos. No. 4,935,233 and 4,751,180. The linker sequence may generally be from 1 to about 50 amino acids in length, e.g., 3, 4, 6, or 10 amino acids in length, but can be 100 or 200 amino acids in length. Linker sequences may not be required when the first and second polypeptides have non-essential N-terminal amino acid regions that can be used to separate the functional domains and prevent steric interference.

Other chemical linkers include carbohydrate linkers, lipid linkers, fatty acid linkers, polyether linkers, e.g., PEG, etc. For example, poly(ethylene glycol) linkers are available from Shearwater Polymers, Inc. Huntsville, Ala. These linkers optionally have amide linkages, sulfhydryl linkages, or heterofunctional linkages.

Other methods of joining the components of the inhibitor and reporter conjugates include ionic binding by expressing negative and positive tails, and indirect binding through antibodies and streptavidin-biotin interactions. (See, e.g. Bioconjugate Techniques, supra). The components can also be joined together through an intermediate interacting sequence. The moieties included in the conjugate molecules can be joined in any order. Example 3 describes a masked inhibitor fusion in which the binding pair member was at the amino terminus, followed by the inhibitor, with the mask at the carboxyl terminus. However, in other instances different orders may be preferable, and no order is uniformly excluded a priori.

Production of Proteins Using Recombinant Techniques

Often, the reporter and inhibitor conjugates included in the system of the invention are protein molecules that are produced by recombinant expression of nucleic acids encoding the proteins as a fusion protein. Expression methodology is well known to those of skill in the art. Such a fusion product can be made by ligating the appropriate nucleic acid sequences encoding the desired amino acid sequences to each other by methods known in the art, in the proper reading frame, and expressing the product by methods known in the art.

Nucleic acids encoding the domains to be incorporated into the fusion proteins of the invention can be obtained using routine techniques in the field of recombinant genetics (see, e.g., Sambrook and Russell, eds, Molecular Cloning: A Laboratory Manual, 3rd Ed, vols. 1-3, Cold Spring Harbor Laboratory Press, 2001; and Current Protocols in Molecular Biology, Ausubel, ed. John Wiley & Sons, Inc. New York, 1997).

Often, the nucleic acid sequences encoding the component domains to be incorporated into the fusion protein are cloned from cDNA and genomic DNA libraries by hybridization with probes, or isolated using amplification techniques with oligonucleotide primers. Amplification techniques can be used to amplify and isolate sequences from DNA or RNA (see, e.g., Dieffenbach & Dveksler, PCR Primers: A Laboratory Manual (1995)). Alternatively, overlapping oligonucleotides can be produced synthetically and joined to produce one or more of the domains. Nucleic acids encoding the component domains can also be isolated from expression libraries using antibodies as probes.

In an example of obtaining a nucleic acid encoding a domain to be included in the conjugate molecule using PCR, the nucleic acid sequence or subsequence is PCR amplified, using a sense primer containing one restriction site and an antisense primer containing another restriction site. This will produce a nucleic acid encoding the desired domain sequence or subsequence and having terminal restriction sites. This nucleic acid can then be easily ligated into a vector containing a nucleic acid encoding the second domain and having the appropriate corresponding restriction sites. The domains can be directly joined or may be separated by a linker, or other, protein sequence. Suitable PCR primers can be determined by one of skill in the art using the sequence information provided in GenBank or other sources. Appropriate restriction sites can also be added to the nucleic acid encoding the protein or protein subsequence by site-directed mutagenesis. The plasmid containing the domain-encoding nucleotide sequence or subsequence is cleaved with the appropriate restriction endonuclease and then ligated into an appropriate vector for amplification and/or expression according to standard methods.

Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Amheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

In some embodiments, it may be desirable to modify the polypeptides encoding the components of the conjugate molecules. One of skill will recognize many ways of generating alterations in a given nucleic acid construct. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, e.g., Giliman and Smith (1979) Gene 8:81-97, Roberts et al. (1987) Nature 328: 731-734.

For example, the domains can be modified to facilitate the linkage of the two domains to obtain the polynucleotides that encode the fusion polypeptides of the invention. Catalytic domains and binding domains that are modified by such methods are also part of the invention. For example, a codon for a cysteine residue can be placed at either end of a domain so that the domain can be linked by, for example, a disulfide linkage. The modification can be performed using either recombinant or chemical methods (see, e.g., Pierce Chemical Co. catalog, Rockford Ill.).

The domains of the recombinant fiusion proteins are often joined by linkers, usually polypeptide sequences of neutral amino acids such as serine or glycine, that can be of varying lengths, for example, about 200 amino acids or more in length, with 1 to 100 amino acids being typical. Often, the linkers are 10, 15, 20, 25, 30, 35, 40, 45, or 50 amino acid residues or less in length. In some embodiments, proline residues are incorporated into the linker to prevent the formation of significant secondary structural elements by the linker. Linkers can often be flexible amino acid subsequences that are synthesized as part of a recombinant fusion protein. Such flexible linkers are known to persons of skill in the art.

In some embodiments, the recombinant nucleic acids encoding the fusion proteins of the invention are modified to provide preferred codons which enhance translation of the nucleic acid in a selected organism (e.g., yeast preferred codons are substituted into a coding nucleic acid for expression in yeast).

Expression Cassettes and Host Cells for Expressing the Fusion polypeptides

There are many expression systems for producing the fusion polypeptide that are well know to those of ordinary skill in the art. (See, e.g., Gene Expression Systems, Fernandes and Hoeffler, Eds. Academic Press, 1999.) Typically, the polynucleotide that encodes the fusion polypeptide is placed under the control of a promoter that is functional in the desired host cell. An extremely wide variety of promoters are available, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, the nucleic acids that encode the joined polypeptides are incorporated for high level expression in a desired host cell.

Expression control sequences that are suitable for use in a particular host cell are often obtained by cloning a gene that is expressed in that cell. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change et al., Nature (1977) 198: 1056), the tryptophan (trp) promoter system (Goeddel et al., Nucleic Acids Res. (1980) 8: 4057), the tac promoter (DeBoer, et al., Proc. Natl. Acad. Sci. U.S.A. (1983) 80:21-25); and the lambda-derived P_{L} promoter and N-gene ribosome binding site (Shimatake et al., Nature (1981) 292: 128). The particular promoter system is not critical to the invention, any available promoter that functions in prokaryotes can be used. Standard bacterial expression vectors include plasmids such as pBR322-based plasmids, e.g., pBLUESCRIPT™, pSKF, pET23D, λ-phage derived vectors, p15A-based vectors (Rose, Nucleic Acids Res. (1988) 16:355 and 356) and fusion expression systems such as GST and LacZ. Epitope tags can also be added to recombinant proteins to provide convenient methods of isolation, e.g., c-myc, HA-tag, 6-His tag, maltose binding protein, VSV-G tag, anti-DYKDDDDK tag, or any such tag, a large number of which ware well known to those of skill in the art.

For expression of fusion polypeptides in prokaryotic cells other than E. coli, regulatory sequences for transcription and translation that finction in the particular prokaryotic species is required. Such promoters can be obtained from genes that have been cloned from the species, or heterologous promoters can be used. For example, the hybrid trp-lac promoter functions in Bacillus in addition to E. coli. These and other suitable bacterial promoters are well known in the art and are described, e.g., in Sambrook et al. and Ausubel et al. Bacterial expression systems for expressing the proteins of the invention are available in, e.g., E. coli, Bacillus sp., and Salmonella (Palva et al., Gene 22:229-235 (1983); Mosbach et al., Nature 302:543-545 (1983). Kits for such expression systems are commercially available.

Similarly, for expression of fusion polypeptides in eukaryotic cells, transcription and translation sequences that function in the particular eukaryotic species are required. For example, eukaryotic expression systems for mammalian cells, yeast, and insect cells are well known in the art and are also commercially available. In yeast, vectors include Yeast Integrating plasmids (e.g., YIp5) and Yeast Replicating plasmids (the YRp series plasmids) and pGPD-2. Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Either constitutive or regulated promoters can be used in the present invention. Regulated promoters can be advantageous because the host cells can be grown to high densities before expression of the fusion polypeptides is induced. High level expression of heterologous proteins slows cell growth in some situations. An inducible promoter is a promoter that directs expression of a gene where the level of expression is alterable by environmental or developmental factors such as, for example, temperature, pH, anaerobic or aerobic conditions, light, transcription factors and chemicals.

For E. coli and other bacterial host cells, inducible promoters are known to those of skill in the art. These include, for example, the lac promoter, the bacteriophage lambda P_{L} promoter, the hybrid trp-lac promoter (Amann et al. (1983) Gene 25: 167; de Boer et al. (1983) Proc. Nat'l. Acad. Sci. USA 80: 21), and the bacteriophage T7 promoter (Studier et al. (1986) J. Mol Biol.; Tabor et al. (1985) Proc. Nat'l. Acad. Sci. USA 82: 1074-8). These promoters and their use are discussed in Sambrook et al., supra.

Inducible promoters for other organisms are also well known to those of skill in the art. These include, for example, the metallothionein promoter, the heat shock promoter, as well as many others.

Translational coupling may be used to enhance expression. The strategy uses a short upstream open reading frame derived from a highly expressed gene native to the translational system, which is placed downstream of the promoter, and a ribosome binding site followed after a few amino acid codons by a termination codon. Just prior to the termination codon is a second ribosome binding site, and following the termination codon is a start codon for the initiation of translation. The system dissolves secondary structure in the RNA, allowing for the efficient initiation of translation. See Squires, et. al. (1988), J. Biol. Chem. 263: 16297-16302.

The construction of polynucleotide constructs generally requires the use of vectors able to replicate in host bacterial cells, or able to integrate into the genome of host bacterial cells. Such vectors are commonly used in the art. A plethora of kits are commercially available for the purification of plasmids from bacteria (for example, EasyPrepJ, FlexiPrepJ, from Pharmacia Biotech; StrataCleanJ, from Stratagene; and, QIAexpress Expression System, Qiagen). The isolated and purified plasmids can then be further manipulated to produce other plasmids, and used to transform cells.

The fusion polypeptides can be expressed intracellularly, or can be secreted from the cell. Intracellular expression often results in high yields. If necessary, the amount of soluble, active fusion polypeptide may be increased by performing refolding procedures (see, e.g., Sambrook et al., supra.; Marston et al., Bio/Technology (1984) 2: 800; Schoner et al., Biol/Technology (1985) 3: 151). Fusion polypeptides of the invention can be expressed in a variety of host cells, including E. coli, other bacterial hosts, yeast, and various higher eukaryotic cells such as the COS, CHO and HeLa cells lines and myeloma cell lines. The host cells can be mammalian cells, insect cells, or microorganisms, such as, for example, yeast cells, bacterial cells, or fungal cells.

Once expressed, the recombinant fusion polypeptides can be purified according to standard procedures of the art, including ammonium sulfate precipitation, affinity columns, column chromatography, gel electrophoresis and the like (see, generally, R. Scopes, Protein Purfication, Springer-Verlag, N.Y. (1982), Deutscher, Methods in Enzymology Vol. 182: Guide to Protein Purification., Academic Press, Inc. N.Y. (1990)). Substantially pure compositions of at least about 90 to 95% homogeneity are preferred, and 98 to 99% or more homogeneity are most preferred.

To facilitate purification of the fusion polypeptides of the invention, the nucleic acids that encode the fusion polypeptides can also include a coding sequence for an epitope or “tag” for which an affinity binding reagent is available. Examples of suitable epitopes include the myc and V-5 reporter genes; expression vectors useful for recombinant production of fusion polypeptides having these epitopes are commercially available (e.g., Invitrogen (Carlsbad Calif.) vectors pcDNA3.1/Myc-His and pcDNA3.1V5-His are suitable for expression in mammalian cells). Additional expression vectors suitable for attaching a tag to the fusion proteins of the invention, and corresponding detection systems are known to those of skill in the art, and several are commercially available (e.g. FLAG” (Kodak, Rochester N.Y.). Another example of a suitable tag is a polyhistidine sequence, which is capable of binding to metal chelate affinity ligands. Typically, six adjacent histidines are used, although one can use more or less than six. Suitable metal chelate affinity ligands that can serve as the binding moiety for a polyhistidine tag include nitrilo-tri-acetic acid (NTA) (Hochuli, E. (1990) “Purification of recombinant proteins with metal chelating adsorbents” In Genetic Engineering: Principles and Methods, J. K. Setlow, Ed., Plenum Press, NY; commercially available from Qiagen (Santa Clarita, Calif.)).

One of skill would recognize that modifications could be made to the protein domains without diminishing their biological activity. Some modifications may be made to facilitate the cloning, expression, or incorporation of a domain into a fusion protein. Such modifications are well known to those of skill in the art and include, for example, the addition of codons at either terminus of the polynucleotide that encodes the binding domain to provide, for example, a methionine added at the amino terminus to provide an initiation site, or additional amino acids (e.g., poly His) placed on either terminus to create conveniently located restriction sites or termination codons or purification sequences.

### Applications of the Systems

The methods and systems of the invention have many applications for which they offer distinct advantages over existing molecular interaction-sensing technologies such as two-hybrid systems and fragment complementation systems. These applications include but are not limited to (1) analyte detection assays for clinical diagnostics, food testing, environmental testing, and process monitoring, (2) high-throughput screening systems for inhibitors of protein-protein interactions involved in disease, (3) epitope-specific selection of antibodies or other binding proteins from libraries, (4) identification of natural ligands of proteins of interest in expressed sequence libraries, (5) engineering enzyme activities for pharmaceutical and industrial applications, and (6) affinity maturation of antibodies and other binding proteins.

Analyte Detection

For example, analyte-mediated disinhibition systems can be used as sensitive and convenient assays to detect the presence of an analyte in clinical, biological, or environmental specimens. Analytes can be small molecules or macromolecules, or even viruses or cells. Such assays are homogeneous and require no manipulations other than mixing the system components with a specimen. In the preferred embodiment the specimen is first equilibrated with a molecule to which the analyte binds (“bait”) fused to the reporter. The bait can be an antibody or scaffolded peptide binder, or it can be a natural ligand of the analyte. The concentration of the bait-reporter fusion should be at least ten times the baitanalyte K_{d} to ensure saturation of the analyte. Then a surrogate for the analyte fused to the inhibitor is added to the mixture. The surrogate can be the analyte itself or any mimic that binds to the same site on the bait, such as a scaffolded peptide isolated using the present invention with the analyte and binder as the binding pair. The surrogate should have an affinity for the bait equal to or greater than that of the analyte. The concentration of the surrogate-inhibitor fusion should be at least ten times that of the bait-reporter fusion to ensure that all reporter fusions that are not bound by analyte are inhibited. Once the mixture is equilibrated, the amount of analyte present will be directly proportional to the reporter activity. If the reporter is an enzyme such as β-lactamase, an excess of chromogenic or fluorogenic substrate can be added, and the activity may be determined spectrophotometrically or fluorimetrically from the first-order rate of color development. Useful substrates for β-lactamase include the chromogen nitrocefin (λmax=485 nm; ε=17,420 M⁻¹ cm⁻¹; McManus-Munoz and Crowder, Biochemistry 38, 1547-53 (1999)) and the fluorogen CCF2/AM (excitation @409 nm, emission @520 nm; Zlokarnik et al., Science 279, 84-88 (1998)).

Target-mediated disinhibition can also be used as a universal platform for the design of biosensors for automated electronic or optical detection and quantification of analytes (Lowe, Philos Trans R Soc Lond B Biol Sci., 324:487-96 (1989). Most current biosensor platforms are quite limited in the types of molecules they can detect. For example, most require enzymatic oxidation or other chemical transformation of the analyte. A few biosensors work by coupling specific analyte binding to the enzymatic generation of an electrical or optical signal, but these are generally not generalizable. Target-mediated disinhibition systems can be set up to couple the binding of any analyte, including small molecules, macromolecules, viruses, and cells, to the generation of electrical or optical signals by using an appropriate enzyme or fluorescent reporter protein and a low-affinity or masked inhibitor of the reporter with analyte binder and surrogate. For example, an oxidase could be linked to the analyte binder and mixed (at ≧10×K_{d}) in an electrode chamber with a sample from a flow stream. The surrogate-inhibitor fusion (surrogates and inhibitors can be isolated as described above) is then added, and after equilibration, an excess of an electron donor substrate of the oxidase is added. The analyte concentration in the flow stream is proportional to the resulting electrical signal.

Inhibitor Selection

In another important application of the target-mediated reporter disinhibition platform interaction-inhibited reporters could be set up and deployed in cells or in vitro for positive selection of inhibitors of key interactions in signal transduction, gene expression, or metabolic pathways. With the completion of the human genome it is expected that thousands of new therapeutic targets will emerge from the elucidation of the protein interaction circuits which underly these processes, and which mis-function in disease. Assays for many of these interactions will be needed for high-throughput screening of synthetic and natural product chemical libraries for inhibitors, which can then be developed into therapeutic drugs. Target binding pairs could be fused to an appropriate reporter and inhibitor and expressed in bacterial, or mammalian cells. β-lactamase and BLIP, for example, would work in both cell types. The reporter gene would be repressed until the cells were exposed to candidate inhibitors, whereupon its expression would be induced, and any reporter activity significantly above background would indicate a potential inhibitor of the interaction. Potential inhibitors would have to be counter-screened in the absence of an interactor to eliminate inhibitors of reporter-inhibitor binding.

Epitope-guided Selection of Antibodies and Other Binding Molecules

The purpose of this application of the system and methods of the invention is to guide the selection of antibodies and other binding molecules to specific antigen epitopes which are functionally relevant, such as the ligand-binding site on a receptor or the epitope of a murine antibody that has desired bioactivity, or to epitopes which are most amenable to high-affinity protein-protein interactions in an aqueous environment. Many murine antibodies have unique bioactivities that are determined primarily by the epitopes they target. However, these antibodies can have limited therapeutic utility in humans. This problem can be overcome by using the murine antibody and antigen in question in an epitope-guided selection system to select fully human antibodies that target the same epitope. In addition to antibodies that bind to desired epitopes on the antigen surface, the epitope “guides” can be scaffolded peptides or other artificial binding proteins, or natural ligands.

In the preferred embodiment the epitope guide is fused to the inhibitor (e.g., BLIP) and the antigen is fused to the reporter (e.g., β-lactamase). This ensures that under conditions in which the reporter is strongly inhibited, the antigen is not in excess. The antigen must be limiting for competition to work properly. However, when the K_{d} of the binding pair interaction is comparable to (within 10% of) or below the working concentrations of the antigen and epitope guide fusion proteins, and the fusion proteins are expressed at comparable levels, then the reporter will be strongly inhibited regardless of whether the antigen is fused to the inhibitor or reporter, etc. In such cases the pairing of fusion partners can be dictated by stability, i.e., which pairs are most stable. In a related embodiment two epitope guides may be used simultaneously, one fused to the reporter and the other fused to the inhibitor, and the antigen is expressed free. This format is useful when antibodies are desired for mulitple epitopes or all epitopes on an antigen. In this format antigen expression can be regulated independently to keep the antigen limiting without reducing reporter expression, and the pairing of fusion partners can be dictated by stability.

In the preferred embodiment the components of the system are expressed in the bacterial periplasm, since antibodies are only expected to fold properly in secretory compartments, where the oxidizing environment is required for disulfide bond formation. Cells expressing the antigen and epitope guide fusions from a single vector are then transformed with a human antibody library. Any human antibody that binds to the same epitope as the mouse antibody will confer selectable antibiotic resistance on the cells by blocking the inhibitor from binding to the enzyme. In addition to antibodies, libraries of other types of molecules may be subjected to epitope-guided selection by reporter disinhibition for epitope-specific binders. These include peptides, scaffolded peptides, other macromolecules such as polysaccharides, carbohydrates, synthetic small molecule libraries, and natural product libraries.

Protein-protein Interaction Mapping

Target-mediated reporter activation by disinhibition has unique utility for the identification of natural interactions among the proteins involved in such findamental cellular processes as signal transduction, gene expression, and regulation of metabolism. The most successful current method for identifying natural ligands of proteins of interest from expressed sequence libraries is the yeast two-hybrid system (Fields and Song, Nature 340:245-247 (1989); Chien et al., Proc. Natl. Acad. Sci. (USA) 88:9578-9582 (1991)). In this method the “bait” protein is fused to a transcription factor DNA-binding domain, and the expressed sequence library is fused to the transactivation domain of the same transcription factor. Both fusion proteins are expressed in yeast cells in which the expression of a reporter gene is dependent on assembly of the transcription factor on upstream DNA, which is in turn dependent on an interaction between the bait protein and a product of the expressed sequence library. Thus, interactors are identified by reporter signal generation. This method suffers from a number of limitations, including high false positive and false negative rates due to (1) the inherent variability of a multistep signal generator, (2) variability due to the broad distribution of stabilities of expressed sequences fused to a meta-stable protein fragment, and (3) the need for heterologous proteins to translocate into and be stable in the alien environment of the yeast cell nucleus. The present invention circumvents most of these difficulties to greatly improve the efficiency of identification of natural protein-protein interactions in expressed sequence libraries.

In one embodiment, a protein of interest (“bait”) is expressed as a fusion to the amino terminus of the reporter, e.g., β-lactamase, and a panel of epitope guides for the bait is expressed as a fusion to the amino terminus of the inhibitor, e.g., BLIP or masked BLIP. The epitope guides may be a panel of thioredoxin-scaffolded peptides or immunoglobulin variable region domains which bind to all available epitopes on the bait, and which could be isolated by any number of methods including phage display (Phage Display of Peptides and Proteins Kay, Winter, and McCafferty, Eds. (1997) Academic Press, San Diego), or the β-lactamase fragment complementation system of U.S. patent application Ser. No. 09/526,106. Typically, both fusion proteins will be expressed from a single vector and the system may be deployed in any appropriate cells, including bacterial, yeast, or mammalian cells. The expressed sequence library is typically derived from randomly-primed, size-selected cDNAs from any desired cell or tissue which is expected to express interactors with the bait protein.

The expressed sequence library genes in expression cassettes on a standard vector are then introduced into the host cells expressing the bait and epitope guide fusions. Generally, each host cell expresses a single epitope guide fusion and a single expressed sequence library member, so that a thorough search would require a number of transformants at least equivalent to the product of the library size by the number of epitope guides. Any expressed sequence product which interacts with the bait, thereby blocking one or more of the epitope guides from docking the inhibitor to the reporter, will be selectable by virtue of the phenotype conferred by the reporter on the host cells, e.g., viability or color. As discussed above for antibody selection, the bait-reporter fusion may be preferred since both the bait and the reporter may need to be limiting to ensure maximum efficiency. However, if the affinities of the epitope guides are high enough the system should work equally well with bait fused to inhibitor and epitope guides fused to the reporter and both expressed at comparable levels.

In an alternative embodiment, the epitope guides may be deployed pair-wise, one fused to the reporter, and the other fused to the inhibitor, and the bait may be expressed free from the same vector. Binding of the guides to separate epitopes on the bait will dock the inhibitor to the reporter. This would reduce by half the number of transformants needed, and would also relax orientation constraints on efficient inhibitor-reporter binding by allowing each epitope guide to pair with a guide which gives the most relaxed orientation on the bait for efficient inhibitor-reporter binding.

Enzyme Engineering

To our knowledge there are no known universal assay platforms for the selection of enzyme variants with altered catalytic activities when neither the parent enzyme(s) nor the desired alterations confer a selectable or screenable phenotype on host cells. The target-mediated reporter disinhibition platform fulfills this need. For any enzyme engineering project, the goal is usually higher catalytic activity for the conversion of one or more specific substrates to one or more specific products. One starts with an enzyme whose properties are as close as possible to those desired, then mutagenizes the gene for the enzyme by any of the known methods, and fmally selects the desired variant from the population of mutagenized enzymes. To detect enzyme variants that have such properties, an assay is required which produces a readout which is proportional to the catalytic rate for the desired substrate-product conversion.

A quantitative product sensor to detect improved enzymes can be fashioned using a target-mediated reporter disinhibition system, a molecule that binds specifically to the desired reaction product, and a surrogate molecule for the desired reaction product. The product binder should discriminate at least 1000-fold against the substrate, and the affinity of the surrogate for the product binder should be comparable to that of the product. In the preferred embodiment the product binder is fused to the reporter and the surrogate is fused to the inhibitor. These two fusions are then co-expressed in the same host cells along with the library of enzyme variants. The cells are then cultivated in the presence of limiting substrate, i.e. comparable to the K_{M} of the parent enzyme. As product is formed it binds to the product binder, competitively inhibiting binding of the surrogate to the product binder. This in turn inhibits docking of the inhibitor to the reporter, thereby activating the reporter.

Under optimal conditions the product binder-reporter fusion is expressed at a level which is equivalent to at least ten times its K_{d} for the product, and the surrogate-inhibitor fusion is co-expressed in the same cells at a level which is comparable to that of the product binder-reporter fusion. Under these conditions the reporter readout should be proportional to the rate of product formation. If the reporter is β-lactamase and the inhibitor is BLIP, the readout could be growth rate in suspension culture in the presence of ampicillin. Since the growth rate of a given cell should be proportional to the activity of the subject enzyme variant it expresses, the culture should become enriched for clones expressing the most active variants. These are then isolated by plating aliquots of the culture on solid medium containing ampicillin at concentrations which are non-permissive for cells expressing the parent enzyme.

U.S. Pat. Nos. 6,294,330; 6,220964; 6,342,345; and U.S. patent application Ser. No. 09/526,106, filed on Mar. 15, 2000 disclose related systems, reporters, binding pairs, methods of use, expression vectors, host cells, etc., and the disclosure of these documents are hereby incorporated by reference.

### EXAMPLES

### Example 1

### Interaction-mediated Inactivation of β-lactamase and Activation by Competitive Disinhibition

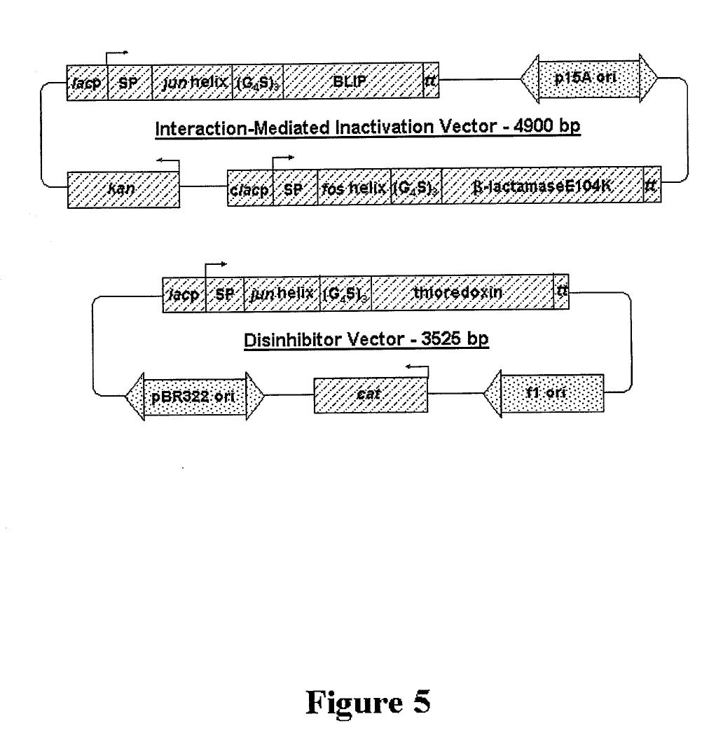

This example demonstrates the use of the methods and systems of the invention. An interaction between the c-fos and c-jun leucine zipper helices (39 amino acids each) was used to ablate β-lactamase activity in E. coli cells by docking an inhibitor, in this example BLIP, to a β-lactamase mutant, E104K, that has reduced affinity for BLIP and cannot therefore be inhibited by BLIP without being docked to it by the heterologous interaction. The expression vectors are illustrated in FIG. 5. The reporter enzyme expression cassette was comprised of a constitutive mutant of the lactose operon UV5 promoter, followed by the coding sequence for a signal peptide for translocation of the fusion protein into the peirplasmic space of the bacterial cell, followed by the c-fos helix fused to the β-lactamase E104K mutant via a (Gly₄Ser)₃ linker. The inhibitor expression cassette was comprised of the lacUV5 promoter, followed by the coding sequence for a signal peptide, followed by the c-jun helix fused to BLIP via a (Gly₄Ser)₃ linker. These cassettes were assembled in a single plasmid based on the p15A replicon (Rose, Nucleic Acids Res. (1988) 16:355 and 356).

The p15A replicon is compatible with pBR322-based vectors and therefore allows the latter to be used for simultaneous expression of the disinhibitor in the same cells. The expression cassette for the disinhibitor was comprised of the lacUV5 promoter, followed by the coding sequence for a signal peptide, followed by the c-jun helix fused to thioredoxin via a (Gly₄Ser)₃ linker. Thioredoxin was used as a stabilizing chaperone for the disinhibitor. A negative control construct for the disinhibitor lacked the coding sequence for the c-jun helix, but otherwise expressed thioredoxin with the amino-terminal (Gly₄Ser)₃ linker. A test construct for a reduced affinity mutant of the disinhibitor was identical to the disinhibitor construct except for a single point mutation which reduced the affinity of the c-jun helix for the c-fos helix by a factor of ˜10. The disinhibitor cassettes were assembled in plasmid pBR322, and all expression vectors were assembled using standard recombinant DNA methods (Sambrook and Russell, eds, Molecular Cloning: A Laboratory Manual, 3rd Ed, vols. 1-3, Cold Spring Harbor Laboratory Press, 2001; and Current Protocols in Molecular Biology, Ausubel, ed. John Wiley & Sons, Inc. New York, 1997). While the β-lactamase fusion was constitutively expressed, expression of the BLIP fusion and disinhibitor genes required IPTG, an inducer of the lacUV5 promoter.

E. coli cells were transformed with these vectors by high-voltage electroporation (Dower et al. (1988) Nucleic Acids Res. 16: 6127-6144), and the transformed cells were plated on solid medium containing various constituents for plasmid maintenance, regulation of the heterologous genes, and to test for antibiotic resistance (Sambrook and Russell, eds, Molecular Cloning: A Laboratory Manual, 3rd Ed, vols. 1-3, Cold Spring Harbor Laboratory Press, 2001). Data reflecting growth of the transformed cells under various conditions are shown in Table I.

When cells expressing the interaction-inhibited β-lactamase vector and any of the disinhibitor vectors were plated on increasing concentrations of ampicillin in the absence of IPTG, so that only the β-lactamase fusion protein was expressed, the cells plated with an efficiency of 100%, i.e., every cell plated produced a colony, on all ampicillin concentrations up to 100 μg/ml. In the presence of IPTG, however, when both the BLIP fusion and disinhibitor were expressed, the negative control cells, expressing thioredoxin without the c-juu helix, did not plate on ampicillin above 10 μg/ml. Since thioredoxin is not expected to interfere with the fosfjun interaction, this represents the maximum fos-jun interaction-mediated inhibition of β-lactamase in the absence of a bona-fide disinhibitor. On 100 μg/ml ampicillin, where plating in the absence of IPTG (no BLIP) was still 100%, up to ten million cells could be plated on IPTG before any colonies appeared. Thus, the maximum signal-to-noise ratio of the system was greater than 1×10⁶.

TABLE I
Interaction-mediated inactivation of β-lactamase and
activation by competitive disinhibition.^{a.}

| | | Ampicillin (μg/ml) | | | |
|---|---|---|---|---|---|
| Disinhibitor^{b.} | IPTG | 10 | 25 | 50 | 100 |
| thioredoxin (trx) | none | 100% | 100% | 100% | 100% |
| c-jun(wt)-trx | none | 100% | 100% | 100% | 100% |
| c-jun(wt)-trx | none | 100% | 100% | 100% | 100% |
| thioredoxin (trx) | 100 μM | 100% | 0.01% | 0.003% | 0.001% |
| c-jun(wt)-trx | 100 μM | 100% | 100% | 60% | 0.002% |
| c-jun(mut)-trx | 100 μM | 100% | 20% | 0.005% | 0.001% |

^{a.}E. coli cells expressing β-lactamase and BLIP as fusions to the c-fos and c-jun leucine zipper helixes, respectively, and further expressing various disinhibitor fusions, were plated on solid medium containing increasing amounts of ampicillin. The data are expressed as % plating efficiency, i.e., % of doubly transformed cells plated which produced colonies after overnight growth at 33° C. The BLIP fusion and the disinhibitors require IPTG for expression.
^{b.}Thioredoxin is the negative control for the disinhibitor. c-jun-trx fusions are the test constructs. wt, wild-type; mut, a point mutant of c-jun that has ˜10-fold lower affinity for c-fos.

When the cells expressed wild-type c-jun as the disinhibitor (fused to trx), 60% of the cells formed colonies when plated on 50 μg/ml ampicillin. However, when 10⁵ of the negative control cells (trx) were plated on 50 μg/ml ampicillin only three colonies appeared. Thus, the signal-to-noise ratio for competitive activation of fos-jun interaction-inhibited β-lactamase by jun-trx was 2×10⁴. This means that the presence of even small amounts of c-jun in the cells could be readily detected by the system. This demonstrates the utility of the system for detection of target molecules. Furthermore, only two colonies appeared when 10⁵ cells expressing the wild-type c-jun disinhibitor were plated on 100 μg/ml ampicillin. Thus, up to 5 logs of plating efficiency remained available for selection of higher-affinity c-jun mutants, if desired, before maximum β-lactamase activity is reached.

When a lower-affinity c-jun mutant was used, the plating efficiency declined to only 20% on 25 μg/ml ampicillin, but was still 200-fold above that of the negative control. On 50 μg/ml ampicillin up to 10⁴ cells expressing the mutant could be plated before colonies appeared. The fact that the wild-type c-jun plated with a 10⁴-fold higher efficiency than a 10-fold lower-affinity point mutant means that the wild type c-jun disinhibitor could have been readily isolated from a large excess of the mutant using this system. This demonstrates the utility of the system for affinity maturation.

### Example 2

### Affinity Maturation of an Antibody Using a Target-mediated β-lactamase Disinhibition System

This example demonstrates the utility of the invention for affinity maturation by demonstrating the selection of a higher-affinity variant of an antibody from a million-fold excess of the parent antibody. The antibody used for this example was a mouse monoclonal raised against the extra-cellular domain of the human B-cell activation antigen CD40, and isolated by hybridoma technology. This antibody, designated HB15, had a K_{d} for CD40 of 7.6 nM, as determined by surface plasmon resonance (Fägerstam et al. (1992) J Chromatog 597: 397-410). A higher-affinity variant of this antibody was subsequently identified which contained two mutations in the third complementarity-determining region (CDR3) of the heavy chain variable region (VH), which conferred a 12-fold increase in the affinity of the antibody. This variant was designated HB15Y.

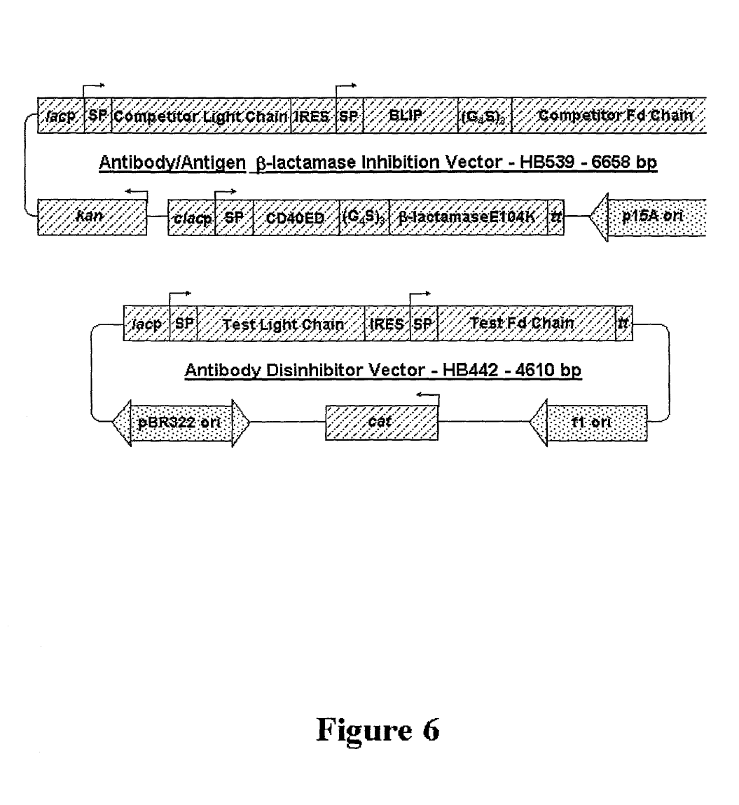

The vectors for expression of the system components for CD40-HB 15 interaction-mediated inhibition of β-lactamase and activation by antibody-mediated disinhibition are depicted in FIG. 6. The CD40-β-lactamaseE104K fusion was expressed from a constitutive mutant of the lacUV5 promoter in the p15A vector denoted HB539. The HB15 antibodies were expressed in Fab form, i.e., VH-CH1 (Fd) with full-length light chain (LC). The Fabs were expressed from dicistronic transcripts driven by the lacUV5 promoter. The upstream cistron encoded the LC, followed by a ribosome binding site (IRES) to allow translation to re-initiate on the downstream cistron, which encoded the Fd fragment. The parent HB15 Fab (competitor) was fused to BLIP at the amino terminus of its Fd fragment via (Gly₄Ser)₃ linker, and expressed from the HB539 vector. The test Fabs were expressed from the pBR322 vector denoted HB442.

A Fab against an irrelevant antigen, i.e., glutathione S-transferase (GST), was used as a negative control. Table II presents data on the ability of these various Fabs to inactivate β-lactamaseE104K by docking BLIP thereto, and on their ability to competitively activate antibody-inhibited β-lactamase. Whereas, host cells expressing the GST Fab as the competitor, i.e., fused to BLIP, plated with 100% efficiency on ampicillin up to 200 μg/ml, the plating efficiency of cells expressing HB15 dropped steadily to <1 colony per 100,000 cells plated on 200 μg/ml ampicillin, indicating specific docking of BLIP to β-lactamase E104K by the HB15-CD40 interaction. The HB15Y mutant, as competitor did not plate appreciably better than the parent HB15, in spite of having a ˜12-fold higher affinity. This indicates that the working concentrations of these antibodies in the cells are well above their K_{d}s, thus affinity is not limiting for β-lactamase inactivation. The same would be true for affinity selection using any interaction-mediated activation system. Thus, non-competitive selection for affinity can only succeed when working concentrations of the antibody are at or below the target K_{d}s, such that affinity remains limiting for the selectable phenotype. For affinity selection at lower target K_{d}s competition must be used between parent and mutant antibodies to allow the affinity of the mutants to be limiting for selectability.

TABLE II
Antibody Affinity Selection by Competitive Disinhibition of β-lactamase^{a.}

| | | | Ampicillin (μg/ml) | | | | |
|---|---|---|---|---|---|---|---|
| Competitor Fab | Test Fab | Test Fab K_{d} | 10 | 25 | 50 | 100 | 200 |
| GST (neg. control) | — | NA | 100% | 100% | 100% | 100% | 100% |
| HB15 | — | NA | 100% | 45% | 1% | 0.01% | <0.001% |
| HB15Y | — | NA | 100% | 20% | 2% | 0.007% | <0.001% |
| HB15 | GST | — | 100% | 15% | 4% | 0.02% | <0.001% |
| HB15 | HB15 | 7.6 nM | 100% | 100% | 100% | 0.2% | 0.01% |
| HB15 | HB15Y | 0.6 nM | 100% | 100% | 80% | 100% | 0.6% |

^{a.}E. coli cells expressing the constructs of FIG. 6 comprising the coding sequences of the indicated Competitor Fabs abd Test Fabs were plated on solid medium containing the indicated concentrations of ampicillin. The data are expressed as % plating efficiency, i.e., % of doubly transformed cells plated which produced colonies after overnight growth at 33° C. The Fab-BLIP fusion and the Test Fab disinhibitors require IPTG for expression.

Cells expressing HB15 as the competitor and GST Fab as the test antibody did not plate appreciably better than with no test antibody, confirming the inability of the GST Fab to compete with HB15 for binding to CD40. However, when HB15 itself was expressed as both competitor and test antibody, in which case it is referred to as the “reference binding pair member”, plating efficiency was substantially increased, up to 25-fold on 50 μg/ml ampicillin, confirming that the CD40-β-lactamase fusion was limiting for competition between the competitor and test antibodies. When the higher-affinity HB15Y variant was used as the test antibody against the parent HB15 competitor, the plating efficiency increased still further compared to the reference antibody. Thus, the plating efficiency of the HB15Y test antibody reached a maximum of 500-fold higher than that of the reference (HB15) on 100 μg/ml ampicillin.

The foregoing results suggest that in a mixed population of test antibodies, the HB15Y test Fab should be enriched 500-fold relative to the reference antibody after each plating. To test this, cells expressing the HB15Y test Fab were mixed with a 10⁶-fold excess of cells expressing the HB15 reference Fab, and 10×10⁶ cells of the mixed population were plated on 100 μg/ml ampicillin. As expected, at least 10,000 colonies were recovered. These colonies were scraped off the plates, resuspended in fresh medium, and quantified by light scattering optical density at 600 nm. The cells were then replated on 100 μg/ml ampicillin at ˜10 cells per original colony, i.e., ˜100,000 cells total. From these cells 116 colonies were recovered. By PCR and signature sequencing, 44 of these colonies were found to be expressing the HB15Y test Fab, 61 were expressing the HB15 reference Fab, and the remainder appeared to have genetic rearrangements of one sort or another giving rise to false positives. Thus, in just two platings the frequency of the higher-affinity variant had increased from 10⁻⁶ to nearly one in two. This clearly demonstrates the utility of β-lactamase activation by competitive disinhibition for antibody affinity maturation, and by extension, for affinity maturation of any binding molecule.

### Example 3

### Isolation of a Low-affinity Cis-inhibiting Mask for BLIP and Demonstration of Its Use in Interaction-mediated Inhibition of β-Lactamase and in Target-mediated Activation of β-Lactamase by Disinhibition

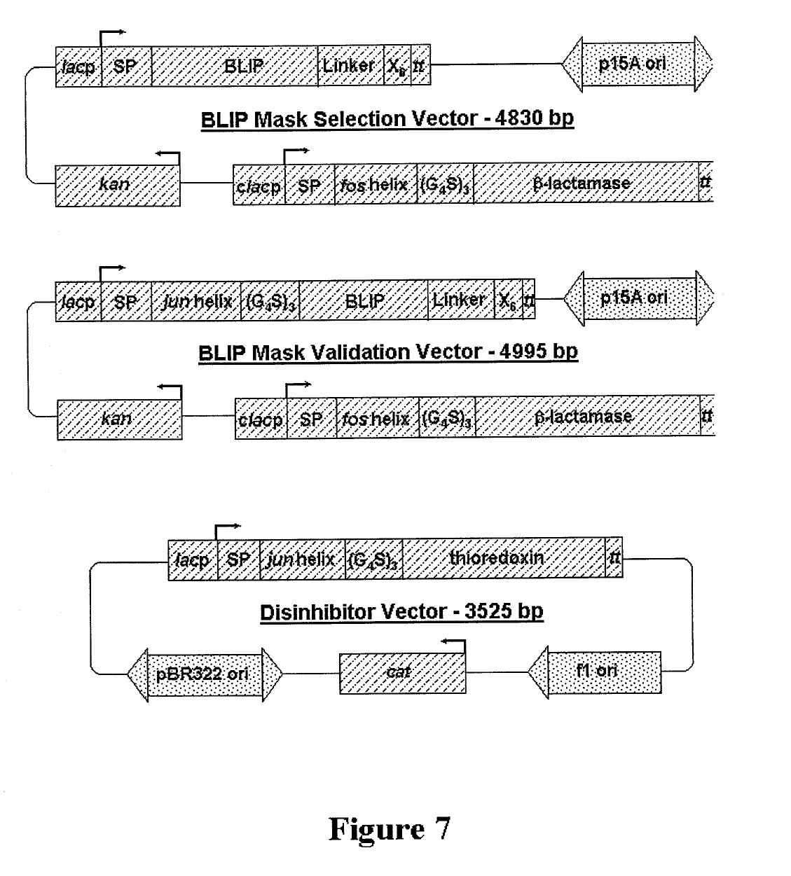

The purpose of this example is to demonstrate the methodology for isolation of low-affinity cis-inhibiting masks for BLIP, and for the use of such masks to improve the efficiency of interaction-mediated β-lactamase inhibition and target-mediated activation by disinhibition. Two key properties are required of the mask to be selected: (1) it must effectively inhibit the subject protein in cis, i.e., when covalently attached to the subject protein, usually by peptide linker, and (2) the mask must be readily displaced when the subject protein is docked to its target or activator. In the case of BLIP the mask must prevent the undocked binding of BLIP to wild-type β-lactamase, while being readily displaced by β-lactamase when the masked BLIP and β-lactamas interaction of binding pair members fused to them. Thus, for optimal mask selection BLIP was expressed from the BLIP Mask Selection Vector depicted in FIG. 7 with an additional six randomly-encoded amino acids linked to its carboxyl terminus via a flexible peptide linker. The random peptide library was encoded by the NNK (ctag, ctag, tg) degenerate codon, which encodes all twenty amino acids but eliminates two of the three stop codons. Wild-type β-lactamase was expressed from the same vector from the constitutive lacUV5 promoter.

When free BLIP and wild-type β-lactamase are expressed in E. coli cells under such conditions β-lactamase is strongly inhibited and the cells do not plate on ampicillin above 10 μg/ml. On 100 μg/ml ampicillin, the plating efficiency is <10⁻⁶. At least 10⁶ library transformants were plated on 100 μg/ml, and the resultant colonies were replated twice at 100 cells per colony. Approximately 30 clones were recovered which consistently plated with high efficiency on 100-200 μg/ml ampicillin. Eighteen of these clones were found not to have genetic rearrangements which ablated BLIP activity. These 18 clones were then tested in the Validation Vector shown in FIG. 7 for the ability to allow BLIP to inhibit β-lactamase when docked to the latter by the fos-jun leucine zipper interaction. Nine clones showed detectable inhibition of β-lactamase when docked by the fos-jun helix interaction. The sequences for these clones and their activities are shown in Table III.

Though all nine selected masks had similar sequences, they varied considerably in their activities. The HB501-1 peptide conferred the greatest degree of dependence on the fos-jun helix interaction, since under the conditions tested BLIP showed essentially no activity in the absence of the interaction, and nearly full activity when docked to β-lactamase by the fos-jun helix interaction. The HB501-1 mask was then tested in the validation vector for the ability to support activation of β-lactamase by competitive disinhibition. This was accomplished by transforming the cells with the same disinhibitor vector used in Example 1 (see FIGS. 5 and 7), which expressed the jun-thioredoxin fusion as the disinhibitor. Under these conditions the plating efficiencies went from 100% on 10 μg/ml ampicillin to 100% on 25 μg/ml, but only 0.1% on 50,μg/ml. Thus, activation by competitive inhibition was successful, though not as strong as when the β-lactamaseE104K mutant was used with the unmasked BLIP. This actually confers an advantage for affinity maturation under these expression conditions, since it leaves a larger proportion of inhibited reporter activity available for recovery by higher-affinity variants, thereby increasing the dynamic range of the system for more sensitive selection of higher affinity variants.

Thus, the utility of low-affinity cis-acting inhibitor masks in interaction- mediated reporter inhibition, and in reporter activation by disinhibition has been demonstrated.

TABLE III
Sequences of Selected BLIP Masks
and their BLIP-reactivation Activities.^{a.}

| | | | Ampₘₐₓ (μg/ml) | |
|---|---|---|---|---|
| | ---------Linker----------- | Mask | +jun | −jun |
| HB501-1 | BLIP-SGGGSGGGNGGGSGGAAAGGGGADIE | ELRLTL | 10 | 200 |
| HB501-2 | ″ ″ | LT | 50 | 100 |
| HB501-3 | ″ ″ | LTPTVN | 50 | 100 |
| HB501-4 | ″ ″ | LTPVTI | 50 | 100 |
| HB501-5 | ″ ″ | LHTVGL | 25 | 100 |
| HB501-6 | ″ ″ | LTLHPT | 25 | 200 |
| HB501-7 | ″ ″ | LLTAAA | 50 | 100 |
| HB501-8 | ″ ″ | LTPT | 50 | 100 |
| HB501-9 | ″ ″ | LTRSLP | 25 | 200 |
| Control | ″ none | none | 10 | 10 |

^{a.}Sequences are listed in single-letter code. Ampₘₐₓ, the maximum ampicillin concentration on which cells plated at ˜100% efficiency while expressing the masked BLIP and wild-β-lactamase either docked together by the fos-jun helix interaction (+jun), or not docked (−jun).

All publications, patents, and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A system for detecting a target molecule that interferes with the binding interaction between members of a binding pair, the system comprising: i) a reporter molecule linked to a first binding pair member, and ii) a low-affinity inhibitor of the reporter molecule linked to a second binding pair member; wherein, when the first and second binding pair members interact, the reporter molecule is inhibited;
and further; wherein binding of the target molecule to at least one binding pair member prevents the inhibitor from binding to the reporter molecule, thereby activating the reporter molecule.

2. The system of claim 1, wherein the low-affinity inhibitor is a scaffolded peptide.

3. The system of claim 2, wherein the scaffolded peptide is a thioredoxin-scaffolded peptide.

4. The system of claim 1, wherein the low-affinity inhibitor is a peptide of between 5 and 20 amino acids.

5. The system of claim 1, wherein the low-affinity inhibitor is an immunoglobulin variable-region domain.

6. The system of claim 1, wherein the interaction between binding pair members is indirect, being mediated by one or more additional molecules.

7. The system of claim 1, wherein the reporter molecule is an enzyme.

8. The system of claim 7, wherein the enzyme is β-lactamase.

9. The system of claim 8, further wherein the low affinity inhibitor is a β-lactamase inhibitor protein (BLIP).

10. The system of claim 1, wherein the binding pair is an antibody and an antigen to which it binds.

11. The system of claim 1, wherein the binding pair is an immunoglobulin variable region domain and a molecule to which it binds.

12. The system of claim 1, wherein the binding pair is a scaffolded peptide and a molecule to which it binds.

13. The system of claim 1, wherein the binding pair is a receptor and a ligand that binds the receptor.

14. The system of claim 1, wherein the reporter molecule is linked to a binding pair member via a linker.

15. The system of claim 1, wherein the inhibitor is linked to the second binding pair member via a linker.

16. A method of detecting a target molecule in a sample, the method comprising:
contacting a sample that is being tested for the presence of the target molecule with: i) a reporter molecule linked to a binding pair member, and ii) a low-affinity inhibitor of the reporter molecule linked to a second binding pair member,
wherein binding of the target molecule to at least one binding pair member prevents the inhibitor from binding to the reporter molecule, thereby activating the reporter molecule.

17. The method of claim 16, wherein the inhibitor is a scaffolded peptide.

18. The method of claim 17, wherein the scaffolded peptide is a thioredoxin-scaffolded peptide.

19. The method of claim 16, wherein the inhibitor is a peptide of between 5 and 20 amino acids.

20. The method of claim 16, wherein the inhibitor is an immunoglobulin variable-region domain.

21. The method of claim 16, wherein the reporter is an enzyme.

22. The method of claim 21, wherein the enzyme is β-lactamase.

23. The method of claim 22, further wherein the β-lactamase low affinity inhibitor is β-lactamase inhibitor protein (BLIP).

24. The method of claim 16, wherein the binding pair is an antibody and antigen to which it binds.

25. The method of claim 16, wherein the binding pair is an immunoglobulin variable region domain and a molecule to which it binds.

26. The method of claim 16, wherein the binding pair is a scaffolded peptide and a molecule to which it binds.

27. The method of claim 16, wherein the binding pair is a receptor and a ligand that binds the receptor.

28. The method of claim 16, wherein the contacting step is performed in vitro.

29. The method of claim 16, wherein the contacting step is performed within a cell.

30. A method of identifying a target molecule in a cell population, the method comprising:
introducing into the population of cells expression vector(s) comprising a nucleic acid sequence encoding a first binding pair member linked to a reporter molecule and further comprising a nucleic acid sequence encoding a second binding pair member linked to an inhibitor of the reporter molecule;
wherein the reporter molecule is inhibited when the binding pair members interact;
culturing the population of cells under conditions in which the first binding pair member linked to the reporter and the second binding pair member linked to the inhibitor are expressed in the presence of a candidate target molecule, wherein a target molecule that binds to at least one binding pair member prevents the inhibitor from binding to the reporter molecule, thereby activating the reporter molecule; and
selecting a cell in which the reporter molecule is active.

31. The method of claim 30, wherein the selecting step comprises selecting a cell in which the reporter molecule is more active than a reference standard of activity.

32. The method of claim 30, wherein the first or the second binding pair member is an antibody.

33. The method of claim 32, further wherein the candidate interceptor molecule is a member of a library of antibodies.

34. The method of claim 32, wherein the first or the second binding pair member is an immunoglobulin variable region domain.

35. The method of claim 32, wherein the first or the second binding pair member is a scaffolded peptide

36. The method of claim 32, further wherein the candidate target molecule is a member of a library of expressed sequences.

37. The method of claim 30, wherein the inhibitor is a scaffolded peptide.

38. The method of claim 37, wherein the scaffolded peptide is a thioredoxin-scaffolded peptide.

39. The method of claim 30, wherein the inhibitor is a peptide of between 5 and 20 amino acids.

40. The method of claim 30, wherein the inhibitor is an immunoglobulin variable-region domain.

41. The method of claim 30, wherein the reporter molecule is an enzyme.

42. The method of claim 30, wherein the marker is a β-lactamase.

43. The method of claim 30, wherein the inhibitor is a β-lactamase inhibitor protein (BLIP).

44. The method of claim 30, wherein the population of cells is a bacterial cell population.

45. The method of claim 44, wherein the bacterial cell population is gram negative.

46. The method of claim 30, wherein the population of cells is a mammalian cell population.

47. The method of claim 30, wherein the population of cells is a yeast cell population.

48. A system for detecting a target molecule that interferes with a binding interaction between members of a binding pair, the system comprising two components:
(i) a reporter molecule linked to a first binding pair member,
(ii) an inhibitor of the reporter molecule linked to a second binding pair member;
wherein component (i) further comprises a mask that has low affinity for the reporter molecule, which mask binds to the reporter molecule and prevents the inhibitor from binding to the reporter molecule when there is no binding pair interaction, or
component (ii) further comprises a mask that has low affinity for the inhibitor, which mask binds to the inhibitor and prevents the inhibitor from binding to the reporter molecule when there is no binding pair interaction,wherein interaction of the binding pair inactivates the reporter by displacement of the mask from the reporter or inhibitor; and further,
wherein binding of the target molecule to the first or second binding pair member prevents the interaction between the first and the second binding pair member, thereby preventing displacement of the mask and binding of the inhibitor to the reporter molecule.
